(19) United States
(12) Patent Application Publication
(10) Pub. No.: US 2022/0218712 A1
(43) Pub. Date: Jul. 14, 2022

(54) TREATMENT OF ALZHEIMER'S DISEASE

(71) Applicants: Saint Louis University, St. Louis, MO (US); U.S. Department of Veterans Affairs, Washington, DC (US)

(72) Inventors: Daniela Salvemini, Chesterfield, MO (US); Susan A. Farr, St. Louis, MO (US)

(21) Appl. No.: 17/433,808

(22) PCT Filed: Feb. 25, 2020
(86) PCT No.: PCT/US2020/019756

§ 371 Date: Aug. 25, 2021

Related U.S. Application Data
(60) Provisional application No. 62/810,068, filed on Feb. 25, 2019.

(51) Int. Cl. A61K 31/52; A61P 25/28

(52) U.S. Cl. CPC A61K 31/52; A61P 25/28

(57) ABSTRACT

Disclosed herein are methods and compositions for the treatment of Alzheimer's disease by administering to a subject in need thereof a selective agonist for the human adenosine A3 receptor (A3AR) subtype. Also disclosed are methods and compositions for prophylactically treating Alzheimer's disease by administering to a subject in need thereof a selective agonist for the human adenosine A3 receptor (A3AR) subtype.

### CROSS REFERENCE

This application is a U.S. national stage application of International Patent Application No. PCT/US2020/019756 filed Feb. 25, 2020, which claims the benefit of U.S. Provisional Application No. 62/810,068 filed Feb. 25, 2019, each of which is entirely incorporated herein by reference for all purposes.

### BACKGROUND

The present disclosure relates to the field of medicine. Specifically, the present disclosure is directed to the use of a drug that is a selective agonist for the human adenosine A3 receptor (A3AR) subtype for the treatment of Alzheimer's disease. Treatment with a selective agonist for the A3AR reverses the memory and cognitive deficits seen with Alzheimer's disease.

Alzheimer's disease (AD) is a chronic neurodegenerative disease that is responsible for 60-70% of all cases of dementia. The most common and earliest symptom is short-term memory loss. This progresses to include problems with language, spatial disorientation, mood swings, loss of motivation, and other problems. Gradually, bodily functions are lost and death ensues. Typical life expectancy following diagnosis is three to nine years. A small percentage of cases are due to inherited factors, but the vast majority are not (“sporadic” AD). AD is a slowly progressive disease. The following stages and their typical durations are generally accepted: mild or early stage (2-4 yr), moderate or middle stage (2-10 yr), and severe or late stage (1-3 yr). At this time a definitive diagnosis of AD requires post mortem examination of the brain. However, a great deal of research is currently searching for reliable biomarkers of disease presence and progress.

The cause of Alzheimer's disease is poorly understood. It is known that abnormal extraneuronal aggregations (plaques) of beta-amyloid (Ab) accumulate in the brain, especially in the hippocampus. Intraneuronal aggregations (tangles) of a hyperphosphorylated form of the protein Tau are also found. In inherited AD, the most common mutations involve the gene that encodes the Ab protein's precursor (APP) and related genes. Postmortem exam shows widespread neurodegeneration (atrophy) throughout the brain. Neuroinflammation and neurodegeneration and are believed to be key factors leading to the cognitive dysfunction associated with AD.

Research on potential treatments for AD use the SAMP8 mouse, which is an inbred mouse strain that spontaneously develops an AD-like dementia that begins during their early adult years. The mice develop deficits in learning and memory starting at approximately 8 months of age and they exhibit an age-related increase in Ab, hyperphosphorylated Tau, oxidative stress, neuroinflammation, and neurodegeneration.

Currently, there are five FDA-approved drugs that may help delay onset, decrease severity or stabilize Alzheimer's symptoms. Four of the five are acetylcholinesterase inhibitors (tacrine, rivastigmine, galantamine and donepezil) and the other (memantine) is an NMDA receptor antagonist. The benefit from their use is small.

It is known that in the brain the purine nucleoside, adenosine, is a major neuroprotective molecule, and that nerve cells, glia and other cell types express receptors on their membranes that have adenosine as their natural ligand. There are known to be four G-protein-coupled receptor subtypes for adenosine: A₁AR, A_{2A}AR, A_{2B}AR, and A₃AR (A3AR). Drug-like molecules are known that have selectivity for binding to each of the four subtypes. In particular, highly-selective (greater than 10,000-fold relative to each of the other three subtypes) agonists for the A3AR are available. Drugs that selectively activate the A3 AR are advantageous because they avoid the cardiovascular, renal and immunological side-effects that are produced by activation of the other three receptor subtypes.

### BRIEF DESCRIPTION

In accordance with the present disclosure, there is provided methods for treating Alzheimer's disease by administering a selective agonist for the human adenosine A3 receptor (MAR) subtype to a patient in need thereof. The selective agonist for the human adenosine A3 receptor subtype (A3AR agonist) may be a compound of Formula (G), (I), (IF), (H), or a compound as described herein below.

Also disclosed are methods for prophylactically treating Alzheimer's disease by administering a selective agonist for the human adenosine A3 receptor (A3AR) subtype to a patient in need thereof.

In certain aspects, the present disclosure provides a compound (or A3AR agonist) of Formula (F):

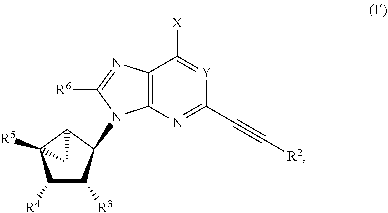

or a pharmaceutically acceptable salt thereof, wherein:
X is selected from NR^{o}R′, CFF, and CH═C(R^{a})(R^{b}),
wherein R^{a} and R^{b} are independently selected from hydrogen, hydroxyl, C₁-C₆ alkyl, and C6-C14 aryl:

### Y is N or CH:

R^{o} is hydrogen or CFF;
R¹ is selected from hydrogen, C₁-C₆ alkyl, C₁-C₆ alkoxy, hydroxyl, C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci₄ aryl C₁-C₆ alkyl, C6-C14 diaryl C1-C6 alkyl, C6-Ci4 aryl C1-C6 alkoxy, heterocyclyl C1-C6 alkyl, heterocyclyl, and C6-C14 aryl C3-C8 cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C6 alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;
R² is selected from C₆-C₁₂ aryl, C₃-C₈ cycloalkyl, heteroaryl, and metallocenyl, wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl;
R³ and R⁴ are independently selected from hydrogen, hydroxyl, amino, mercapto, ureido, C₁-C₆ alkyl carbonylamino, hydroxy C₁-C₆ alkyl, and hydrazinyl;
R⁵ is selected from C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C6 cycloalkylamino, hydroxylamino, and C₂-C₃ alkenyl; and
R⁶ is selected from hydrogen, C₁-C₆ alkyl, C₂-C₆ alkenyl, C₂-C₆ alkynyl, heteroaryl, and Ci-C6 aminoalkyl.

In certain embodiments for a compound of formula (I), wherein when R¹ is methyl, then R² is selected from the group consisting of

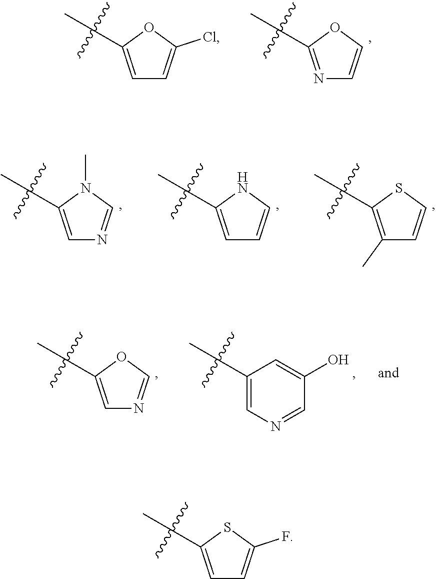

In certain embodiments for a compound of formula (I), wherein when R¹ is halobenzyl or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylmethyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

In certain aspects, the present disclosure provides a compound (or A3AR agonist) of Formula (IF):

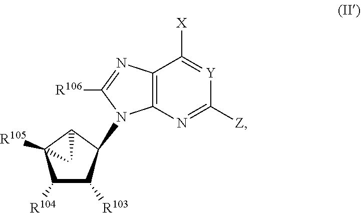

or a pharmaceutically acceptable salt thereof, wherein:
X is selected from NHR¹⁰¹, CFF, and CH═C(R^{a})(R^{b}),
wherein R^{a} and R are independently selected from hydrogen, hydroxyl, C₁-C₆ alkyl, and C6-C14 aryl;

### Y is N or CH;

R¹⁰¹ is selected from C1-C₆ alkyl, C1-C₆ alkoxy, hydroxyl, C3-C8 cycloalkyl, C3-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl C₁-C₆ alkyl, C₆-C₁₄ diaryl C₁-C₆ alkyl, C₆-Ci₄ aryl C₁-C₆ alkoxy, heterocyclyl C₁-C₆ alkyl, heterocyclyl, and C₆-Ci₄ aryl C₃-C8 cycloalkyl,
wherein the aryl or heterocyclyl portion of R¹⁰¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C₂-C₆ alkynyl; and
wherein the alkyl or cycloalkyl portion of R¹⁰¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;

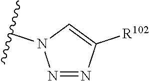

Z is halo, azido, or a group of the formula: N═N wherein:
R¹⁰² is selected from C₆-Ci2 aryl, C₆-C12 aryl-Ci-C6 alkyl, C3-C8 cycloalkyl, heteroaryl, and metallocenyl,
wherein the aryl or heteroaryl group of R¹⁰² is optionally substituted with one or more substituents each independently selected from halo, trifluoromethyl, amino, alkyl, hydroxyalkyl, aryl, alkoxy, hydroxyl, carboxyl, sulfonyloxy, carboxyalkyl, sulfonyloxyalkyl, alkylcarbonyl, and arylcarbonyl;
R¹⁰³ and R¹⁰⁴ are independently selected from hydrogen, hydroxyl, amino, mercapto, ureido, C₁-C₆ alkyl carbonylamino, hydroxy C₁-C₆ alkyl, and hydrazinyl;
R¹⁰⁵ is selected from hydrogen, C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C₆ cycloalkylamino, hydroxylamino, and C₂-C₃ alkenyl; and
R¹⁰⁶ is selected from hydrogen, C₁-C₆ alkyl, C₂-C₆ alkenyl, C₂-C₆ alkynyl, heteroaryl, and Ci-C₆ aminoalkyl.

It is contemplated that any method or composition described herein can be implemented with respect to any other method or composition described herein.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the effects of a selective A3AR agonist, MRS5980, on learning and memory in 12 month-old SAMP8 mice. The selective A3AR agonist, MRS5980 (1 mg/kg, IP) or vehicle (saline) was given every 48 hours for 5 weeks starting at 12 months of age. Behavioral testing began after 3 weeks of treatment. NORPT—the Novel Object Recognition Protocol Test is a memory task that involves the hippocampus when, as performed herein, the retention interval is 24 hours after initial exposure to the two like objects. Mice received three 5 minutes habituation trials in an empty apparatus separated by 2 hours, the day prior to entry of the objects. During the training session, the mouse was exposed to two similar objects (plastic frogs) which it was allowed to examine for 5 minutes. The apparatus and the objects were cleaned between each trial to remove odor cures. Twenty-four hours later, the mouse was exposed to one of the original objects and a new novel object (plastic bird) and the time spent examining the objects was recorded. The results are expressed as Discrimination index=(Total new−Total old)/Total new+Total old)]. The novel object was made out of the same material as the original object and of the same size, but a different shape. The underlying concept of the NORPT is based on the tendency of mice to spend more time exploring new objects than familiar ones. Thus, the greater the retention/memory at 24 hours, the more time spent with the new object the higher the discrimination. Old mice treated with the A3AR agonist demonstrated significantly better memory than vehicle treated controls. There was no difference between the young mice and the old A3 AR treated mice. Number of attempts before first successful avoidance of the shock in T-maze and retention (memory) for the T-maze one-week later was also determined. Retention was defined as number of trial to successfully avoid the shock 5 out of 6 consecutive trials. Old mice treated with the A3 AR agonist learned and remembered the T-maze better than the old vehicle treated controls. There was no difference between the young vehicle treated controls and the old A3AR treated mice. ANOVA followed by pairwise Tukey's post hoc test. Means±SEM; n=6-8/group; **p<0.01.

FIG. 2 depicts the effects of a selective A3 AR agonist, MRS5980, on activity and anxiety in 12 month-old SAMP8 mice. The selective A3AR agonist, MRS5980 (1 mg/kg, IP) or vehicle (saline) was given every 48 hours for 5 weeks starting at 12 months of age. Behavioral testing began after 3 weeks of treatment. Activity in the open field test is a measure of general activity. Mice were allowed to explore a novel open field for 5 minutes. Distance travelled was recorded using an ANY-maze (San Diego Instruments, San Diego, Calif.). The test detects any effects of drug on general activity that may contribute to differences in learning and memory. There were no differences between any of the groups indicating that increased/decreased activity did not contribute to changes in memory. Total time spent in the open arms of an elevated plus maze was determined. Mice that are anxious will spend less time in the open arms. Differences in anxiety can affect learning and memory. There were no statistically significant differences between the groups in the elevated plus maze indicating that anxiety was not a factor in learning and memory differences.

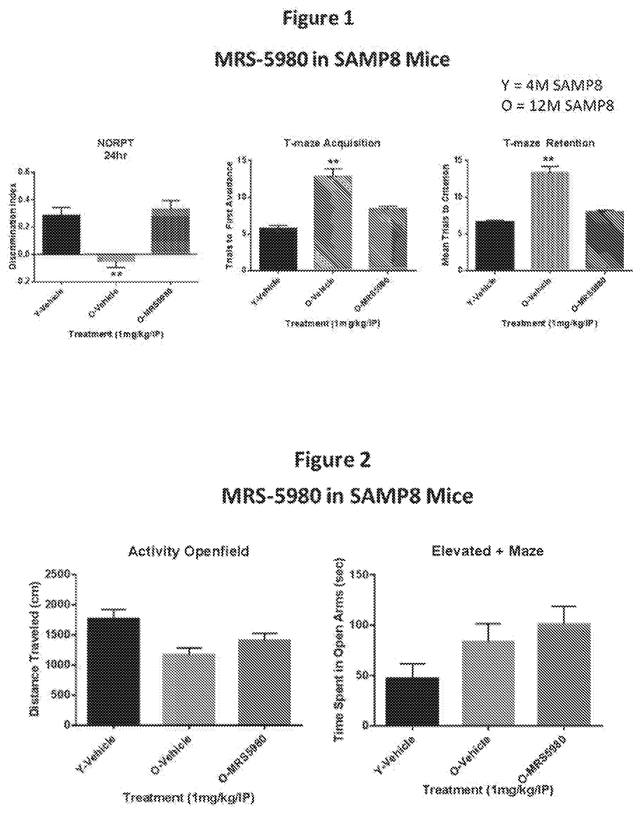

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

As used herein, “a subject in need thereof” refers to a subject susceptible to or at risk of a specified disease, disorder, or condition. More particularly, in the present disclosure the methods of treating Alzheimer's Disease is to be used with a subset of subjects who are susceptible to or at elevated risk for experiencing or developing Alzheimer's Disease. Subjects may be susceptible to or at elevated risk for experiencing or developing Alzheimer's Disease due to family history, age, environment, and/or lifestyle. The methods for prophylactically treating Alzheimer's Disease to be used with a subset of subjects who are susceptible to or at elevated risk for developing Alzheimer's Disease. Subjects who are susceptible to or at elevated risk for developing Alzheimer's Disease include subjects having a family history of Alzheimer's Disease and/or subjects who are determined to have one or more biomarkers associated with having Alzheimer's Disease and/or associated with developing Alzheimer's Disease.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

In one aspect, the present disclosure is directed to methods for treating Alzheimer's Disease by administering a selective agonist for the adenosine A3 human receptor subtype to a patient in need thereof. The present disclosure is also directed to prophylactically treating Alzheimer's Disease by administering a selective agonist for the adenosine A3 human receptor subtype to a patient in need thereof.

It can be confirmed that a compound is a selective A3AR agonist using known methods, including competitive radioimmunoassays and assays of forskolin-stimulated cyclic adenosine monophosphate (cAMP) production in human A3AR transfected CHO cells or HEK cells. “Selective” is herein defined as binding affinity (or cAMP production) for the human A3 receptor subtype that is at least 50-fold greater than the binding affinity (or cAMP production) for any of the other three human receptor subtypes. It is important to specify selectivity with respect to the human form of the A3AR because agonists are known to have significantly different binding affinities for A3AR's from other species.

Suitable selective agonists for the human A3AR may be chosen from, but not limited to, any of the following: (i) N⁶-benzyladenosine-5′-N-methyluronamides such as N⁶-(3-iodobenzyl)-adenosine-5′-N-methyluronamide (also known as IB-MECA), and 2-Chloro-N⁶-(3-iodobenzyl)-adenosine-5′-N-methyluronamide (also known as 2-CI-IB-MECA); (ii) (N)-methanocarba nucleosides such as (1R,2R,3S,4R)-4-(2-chloro-6-((3-chlorobenzyl)amino)-9H-purin-9-yl)-2,3-di-hydroxy-N-methylbicyclo[3.1.0]hexane-1-carboxamide (also known as CF502, Can-Fite Biopharma, MA); (2S,3S,4R,5R)-3-amino-5-[6-(2,5-dichlorobenzylamino)purin-9-yl]-4-hydroxy-tetrahydrofuran-2-carboxylicacid methylamide (also known as CP-532,903); (rS,2′R,3'S,4′R,5'S)-4-(2-chloro-6-(3-chlorobenzylamino)-9H-purin-9-yl)-2, 3-dihydroxy-N-methylbicyclo[3.1.0]hexane-1-carboxamide (also known as MRS-3558); (rR,2′R,3'S,4′R,5'S)-4-{2-chloro-6-[(3-iodophenylmethyl)amino]purin-9-yl-}-1-(methylaminocarbonyl)-bicyclo[3.1.0]hexane-2,3-diol (also known as MRS 1898); and (iii) 2-Dialkynyl derivatives of (N)-methanocarba nucleosides, 2-(arylethynyl)adenine and N(6)-methyl or N(6)-(3-substituted-benzyl), N(6)-methyl 2-(halophenylethynyl) analogues, polyaromatic 2-ethynyl N(6)-3-chlorobenzyl analogues, such as 2-p-biphenylethynyl MRS5679 and fluorescent 1-pyrene adduct MRS 5704, as well as MRS 5678.

Particularly suitable highly-selective (<10,000-fold) A3AR agonists for use in the methods, include but are not limited to, the adenosine methanocarba derivatives described in Tosh et al. (2014; 2015a, 2015b, 2015c, and 2016). In certain embodiments, A3AR agonists may be selected from a compound described in any one of U.S. Pat. Nos. 9,963,450; 8,916,570; 8,735,407; 8,796,291; 9,181,253; and 9,123,131; and US Patent Application No. 20170002007, the compounds and chemical genuses of each of which are incorporated herein by reference. Also included herein by reference are the compounds and chemical genuses described in Publication No. WO2015080940. These compounds include but are not limited to those designated MRS5980, MRS7144, MRS7154, MRS7334, MRS7137, MRS7555, MRS7556, and MRS7557.

In some embodiments, the A3AR agonist is not a compound listed in Tables 1 through 4, or a salt thereof. In some embodiments, the A3AR agonist is not a subset of the compounds and salts thereof listed in Tables 1 through 4. In some embodiments, the A3AR agonist is not a compound listed in Tables 2 through 4, or a salt thereof. In some embodiments, the A3AR agonist is not a subset of the compounds and salts thereof listed in Tables 2 through 4. In some embodiments, the A3AR agonist is not a compound listed in Table 1, or a salt thereof. In some embodiments, the A3AR agonist is not a subset of the compounds and salts thereof listed in Table 1. In some embodiments, the A3AR agonist is not a compound listed in Table 2, or a salt thereof. In some embodiments, the A3AR agonist is not a subset of the compounds and salts thereof listed in Table 2. In some embodiments, the A3AR agonist is not a compound listed in Table 3, or a salt thereof. In some embodiments, the A3AR agonist is not a subset of the compounds and salts thereof listed in Table 3. In some embodiments, the A3AR agonist is not a compound listed in Table 4, or a salt thereof. In some embodiments, the A3AR agonist is not a subset of the compounds and salts thereof listed in Table 4.

TABLE 1
Representative A3AR agonists

| Cmpd # | Structure |
|---|---|
| 1-1 | 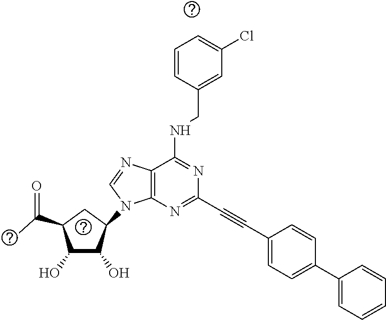 |
| 1-2 | 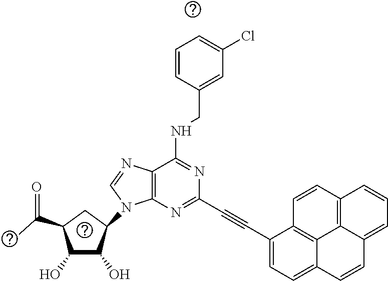 |
| 1-3 | 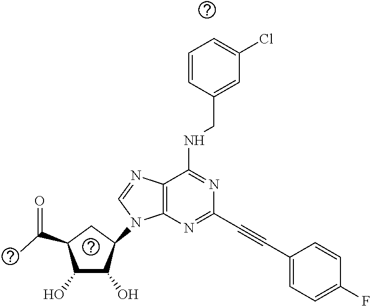 |
| 1-4 | 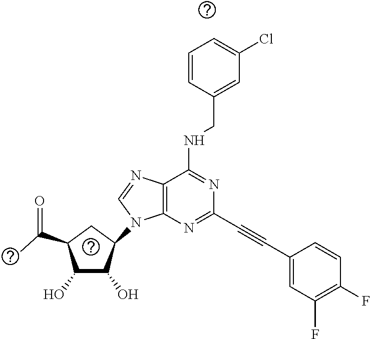 |
| 1-5 | 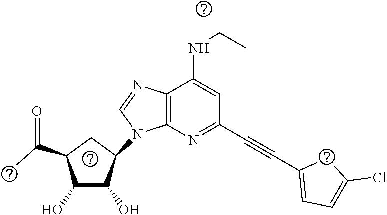 |
| 1-6 | 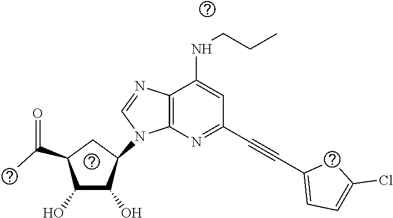 |

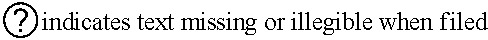

In some embodiments, the A3AR agonist is not a compound or a salt thereof, or any subset of the compounds and salts thereof, listed in Table 1. In some embodiments, the A3 AR agonist is not 1-1 of Table 1, or a salt thereof. In some embodiments, the A3AR agonist is not 1-2 of Table 1, or a salt thereof. In some embodiments, the A3AR agonist is not 1-3 of Table 1, or a salt thereof. In some embodiments, the A3AR agonist is not 1-4 of Table 1, or a salt thereof. In some embodiments, the A3AR agonist is not 1-5 of Table 1, or a salt thereof. In some embodiments, the A3AR agonist is not 1-6 of Table 1, or a salt thereof.

In some embodiments, the A3AR agonist is a compound listed in Table 1, or a salt thereof, or any combination of the compounds or salt thereof listed in Table 1. In some embodiments, the A3AR agonist is 1-1 of Table 1, or a salt thereof. In some embodiments, the A3AR agonist is 1-2 of Table 1, or a salt thereof. In some embodiments, the A3AR agonist is 1-3 of Table 1, or a salt thereof. In some embodiments, the A3AR agonist is 1-4 of Table 1, or a salt thereof. In some embodiments, the A3AR agonist is 1-5 of Table 1, or a salt thereof. In some embodiments, the A3AR agonist is 1-6 of Table 1, or a salt thereof. In some embodiments, the A3AR agonist is not a compound or a salt thereof, or any subset of the compounds and salts thereof, listed in Tables 2 through 4.

TABLE 2
Representative A3 AR agonists

| Cmpd # | Structure |
|---|---|
| II-1 | 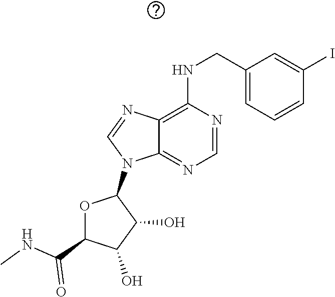 [SMILES: CNC(=O)[C@H]1O[C@@H](n2cnc3c(NCc4cccc(I)c4)ncnc32)[C@H](O)[C@@H]1O] |
| II-2 | 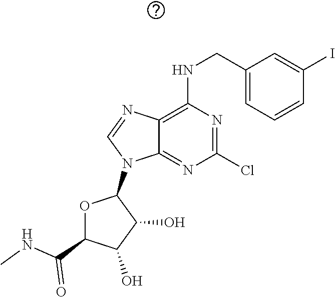 [SMILES: CNC(=O)[C@H]1O[C@@H](n2cnc3c(NCc4cccc(I)c4)nc(Cl)nc32)[C@H](O)[C@@H]1O] |
| II-3 | 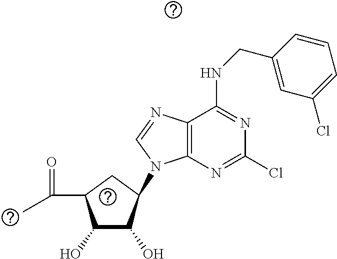 |
| II-4 | 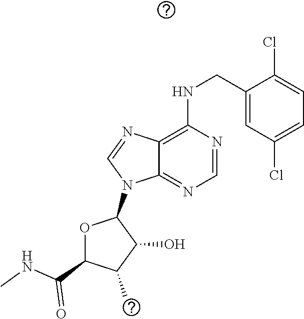 [SMILES: CNC(=O)[C@H]1O[C@@H](n2cnc3c(NCc4cc(Cl)ccc4Cl)ncnc32)[C@H](O)[C@@H]1C] |
| II-5 | 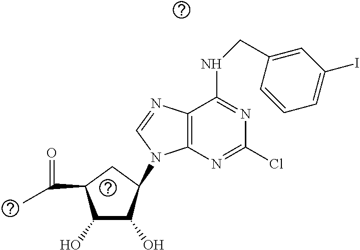 |
| II-6 | 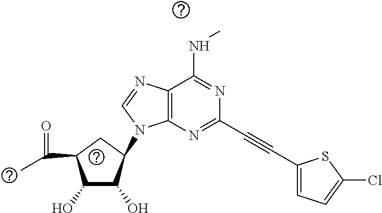 |
| II-7 | 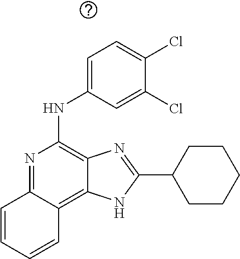 [SMILES: Clc1ccc(Nc2nc3ccccc3c3[nH]c(C4CCCCC4)nc23)cc1Cl] |
| II-8 | 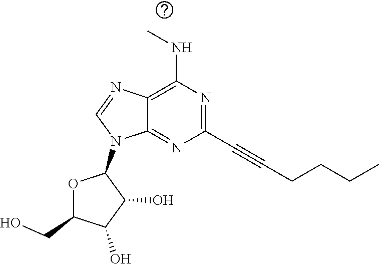 [SMILES: CCCCC#Cc1nc(NC)c2ncn([C@@H]3O[C@H](CO)[C@@H](O)[C@H]3O)c2n1] |
| II-9 | 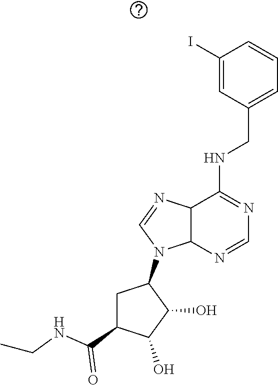 [SMILES: CCNC(=O)[C@H]1C[C@@H](N2C=NC3C(NCc4cccc(I)c4)=NC=NC32)[C@H](O)[C@@H]1O] |
| II-10 | 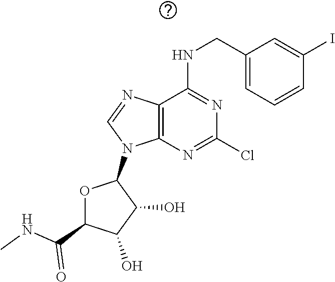 [SMILES: CNC(=O)[C@H]1O[C@@H](n2cnc3c(NCc4cccc(I)c4)nc(Cl)nc32)[C@H](O)[C@@H]1O] |

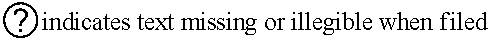

In some embodiments, the A3AR agonist is not a compound or a salt thereof, or any subset of the compounds and salts thereof, listed in Table 2. In some embodiments, the A3AR agonist is not II-1 of Table 2, or a salt thereof. In some embodiments, the A3AR agonist is not II-2 of Table 2, or a salt thereof. In some embodiments, the A3AR agonist is not II-3 of Table 2, or a salt thereof. In some embodiments, the A3AR agonist is not II-4 of Table 2, or a salt thereof. In some embodiments, the A3AR agonist is not II-5 of Table 2, or a salt thereof. In some embodiments, the A3AR agonist is not II-6 of Table 2, or a salt thereof. In some embodiments, the A3AR agonist is not II-7 of Table 2, or a salt thereof. In some embodiments, the A3AR agonist is not II-8 of Table 2, or a salt thereof. In some embodiments, the A3AR agonist is not II-9 of Table 2, or a salt thereof.

In some embodiments, the A3AR agonist is a compound listed in Table 2, or a salt thereof, or any combination of the compounds or salt thereof listed in Table 2. In some embodiments, the A3AR agonist is II-1 of Table 2, or a salt thereof. In some embodiments, the A3AR agonist is II-2 of Table 2, or a salt thereof. In some embodiments, the A3AR agonist is II-3 of Table 2, or a salt thereof. In some embodiments, the A3AR agonist is II-4 of Table 2, or a salt thereof. In some embodiments, the A3 AR agonist is II-5 of Table 2, or a salt thereof. In some embodiments, the A3AR agonist is II-6 of Table 2, or a salt thereof. In some embodiments, the A3AR agonist is II-7 of Table 2, or a salt thereof. In some embodiments, the A3AR agonist is II-8 of Table 2, or a salt thereof. In some embodiments, the A3 AR agonist is II-9 of Table 2, or a salt thereof.

TABLE 3
Representative A3AR agonists

| Cmpd # | Structure |
|---|---|
| III-1 | 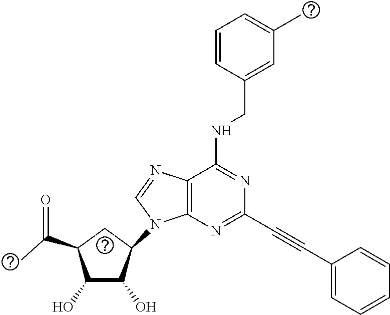 |
| III-2 | 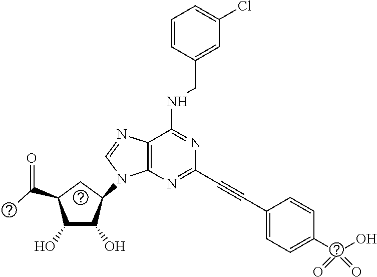 |
| III-3 | 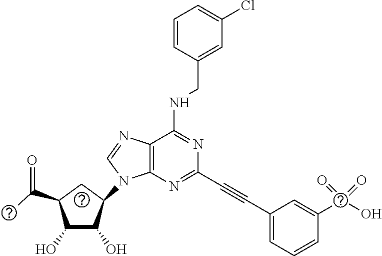 |
| III-4 | 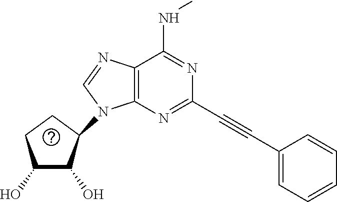 |
| III-5 | 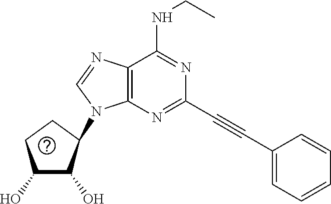 |
| III-6 | 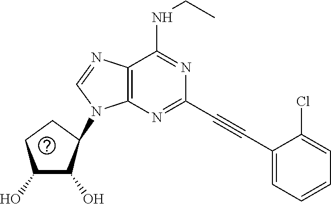 |
| III-7 | 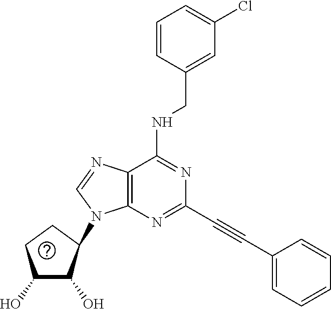 |
| III-8 | 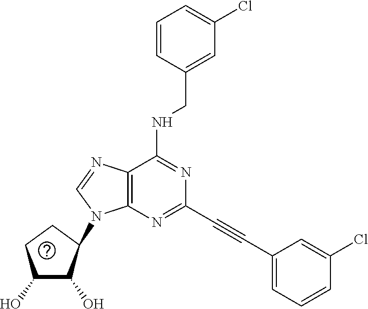 |
| III-9 | 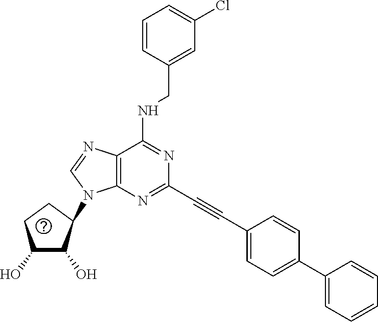 |
| III-10 | 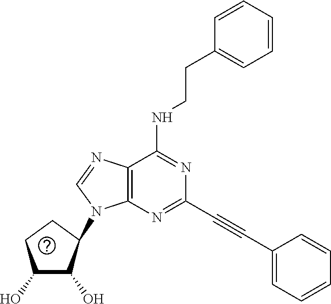 |
| III-11 | 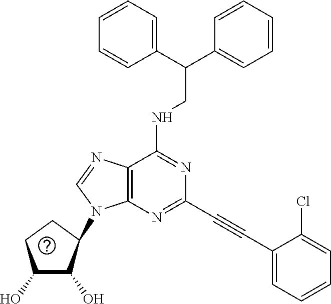 |
| III-12 | 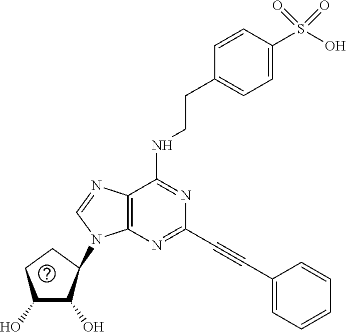 |
| III-13 | 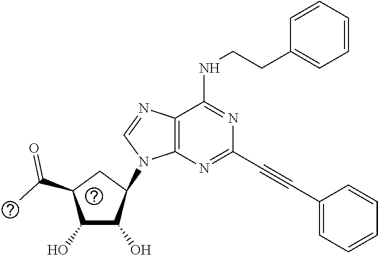 |
| III-14 | 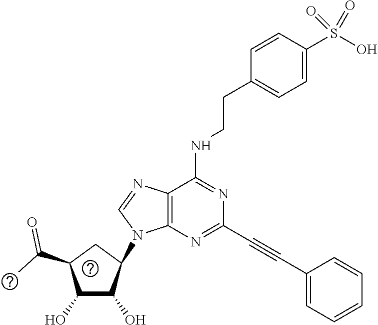 |

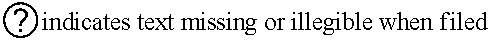

In some embodiments, the A3AR agonist is not a compound or a salt thereof, or any subset of the compounds and salts thereof, listed in Table 3. In some embodiments, the A3AR agonist is not III-1 of Table 3, or a salt thereof. In some embodiments, the A3AR agonist is not III-2 of Table 3, or a salt thereof. In some embodiments, the A3AR agonist is not III-3 of Table 3, or a salt thereof. In some embodiments, the A3AR agonist is not III-4 of Table 3, or a salt thereof. In some embodiments, the A3AR agonist is not III-5 of Table 3, or a salt thereof. In some embodiments, the A3AR agonist is not III-6 of Table 3, or a salt thereof. In some embodiments, the A3AR agonist is not III-7 of Table 3, or a salt thereof. In some embodiments, the A3AR agonist is not III-8 of Table 3, or a salt thereof. In some embodiments, the A3AR agonist is not III-9 of Table 3, or a salt thereof. In some embodiments, the A3AR agonist is not III-10 of Table 3, or a salt thereof. In some embodiments, the A3AR agonist is not III-11 of Table 3, or a salt thereof. In some embodiments, the A3AR agonist is not III-12 of Table 3, or a salt thereof. In some embodiments, the A3AR agonist is not III-13 of Table 3, or a salt thereof. In some embodiments, the A3AR agonist is not III-14 of Table 3, or a salt thereof.

In some embodiments, the A3AR agonist is a compound listed in Table 3, or a salt thereof, or any combination of the compounds or salt thereof listed in Table 3. In some embodiments, the A3AR agonist is III-1 of Table 3, or a salt thereof. In some embodiments, the A3AR agonist is III-2 of Table 3, or a salt thereof. In some embodiments, the A3AR agonist is III-3 of Table 3, or a salt thereof. In some embodiments, the A3AR agonist is III-4 of Table 3, or a salt thereof. In some embodiments, the A3AR agonist is III-5 of Table 3, or a salt thereof. In some embodiments, the A3AR agonist is III-6 of Table 3, or a salt thereof. In some embodiments, the A3AR agonist is III-7 of Table 3, or a salt thereof. In some embodiments, the A3AR agonist is III-8 of Table 3, or a salt thereof. In some embodiments, the A3AR agonist is III-9 of Table 3, or a salt thereof. In some embodiments, the A3AR agonist is III-10 of Table 3, or a salt thereof. In some embodiments, the A3AR agonist is III-11 of Table 3, or a salt thereof. In some embodiments, the A3AR agonist is III-12 of Table 3, or a salt thereof. In some embodiments, the A3AR agonist is III-13 of Table 3, or a salt thereof. In some embodiments, the A3AR agonist is III-14 of Table 3, or a salt thereof.

TABLE 4
Representative A3AR agonists

| Cmpd # | Structure |
|---|---|
| IV-1 | 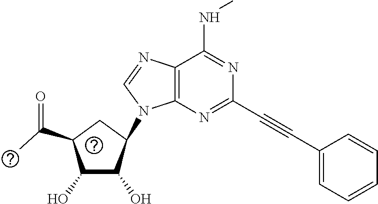 |
| IV-2 | 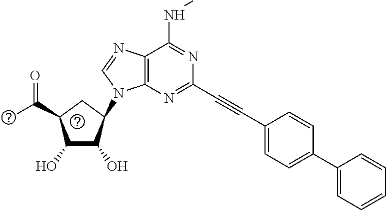 |
| IV-3 | 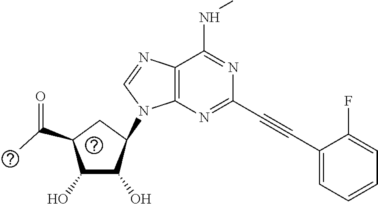 |
| IV-4 | 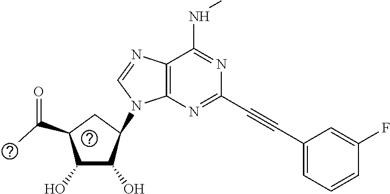 |
| IV-5 | 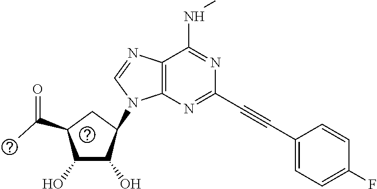 |
| IV-6 | 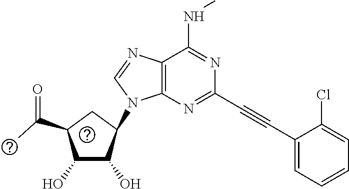 |
| IV-7 | 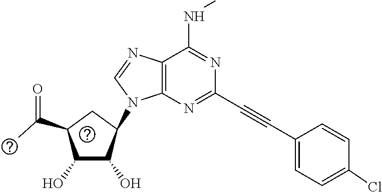 |
| IV-8 | 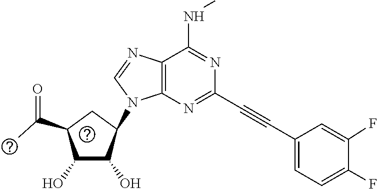 |
| IV-9 | 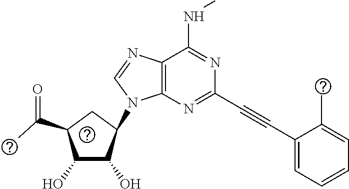 |
| IV-10 | 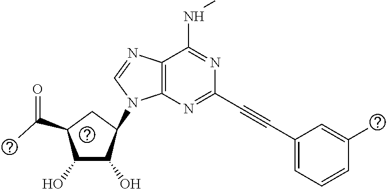 |
| IV-11 | 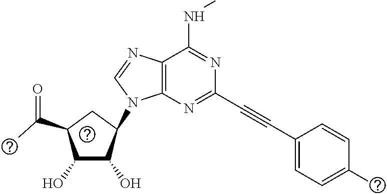 |
| IV-12 | 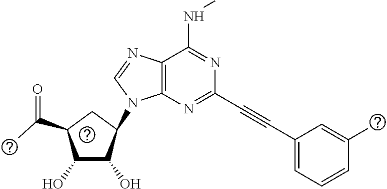 |
| IV-13 | 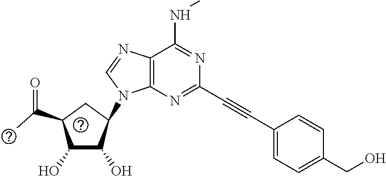 |
| IV-14 | 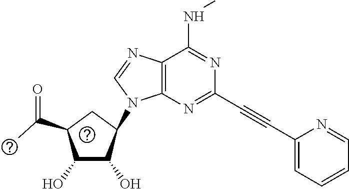 |
| IV-15 | 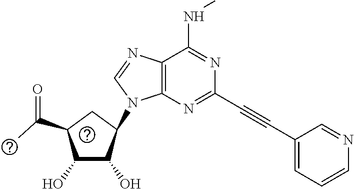 |
| IV-16 | 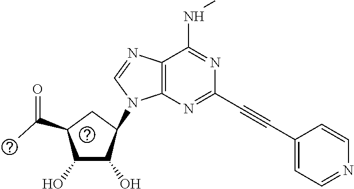 |
| IV-17 | 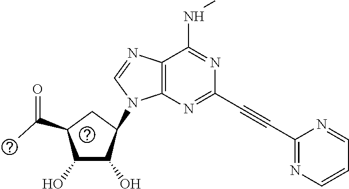 |
| IV-18 | 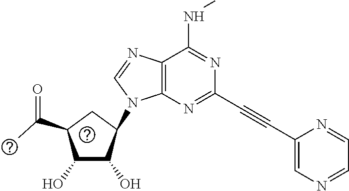 |
| IV-19 | 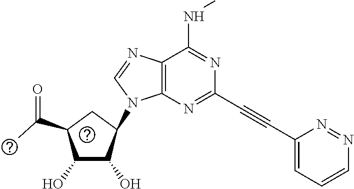 |
| IV-20 | 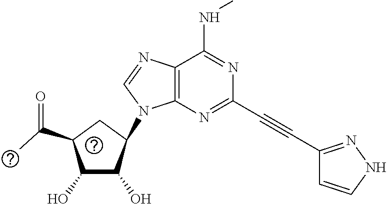 |
| IV-21 | 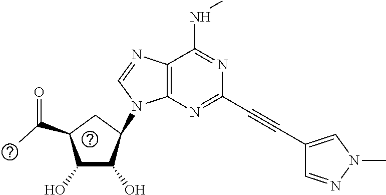 |
| IV-22 | 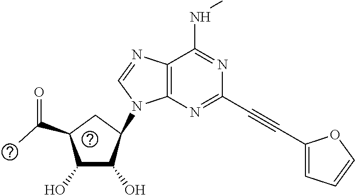 |
| IV-23 | 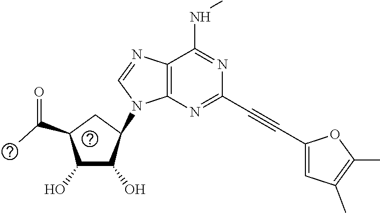 |
| IV-24 | 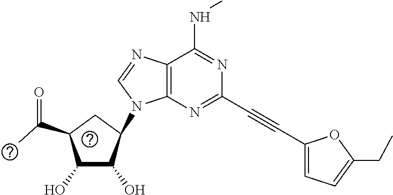 |
| IV-25 | 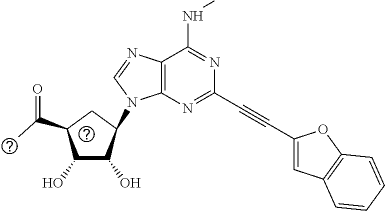 |
| IV-26 | 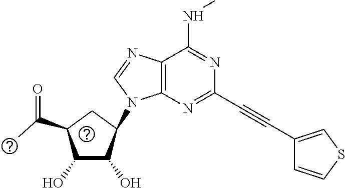 |
| IV-27 | 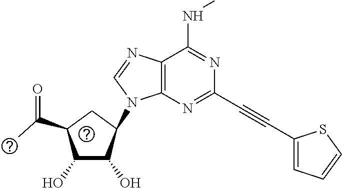 |
| IV-28 | 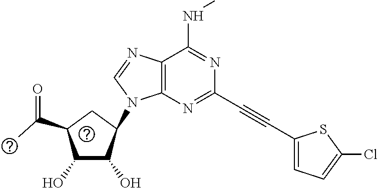 |
| IV-29 | 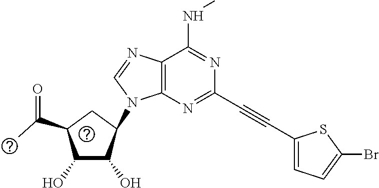 |
| IV-30 | 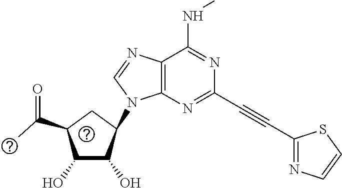 |
| IV-31 | 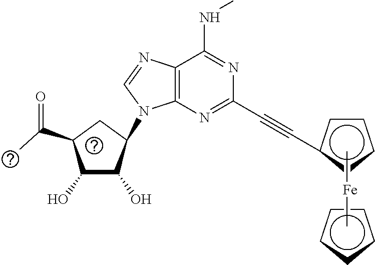 |
| IV-32 | 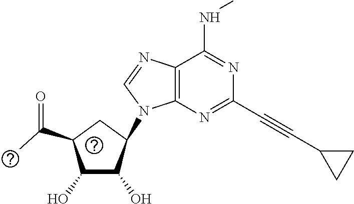 |
| IV-33 | 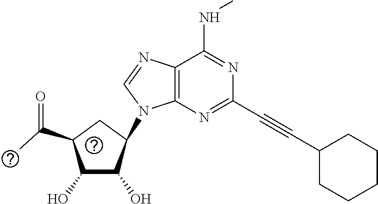 |

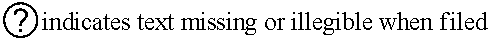

In some embodiments, the A3AR agonist is not a compound or a salt thereof, or any subset of the compounds and salts thereof, listed in Table 4. In some embodiments, the A3AR agonist is not IV-1 of Table 4, or a salt thereof. In some embodiments, the A3AR agonist is not IV-2 of Table 4, or a salt thereof. In some embodiments, the A3AR agonist is not IV-3 of Table 4, or a salt thereof. In some embodiments, the A3AR agonist is not IV-4 of Table 4, or a salt thereof. In some embodiments, the A3AR agonist is not IV-5 of Table 4, or a salt thereof. In some embodiments, the A3AR agonist is not IV-6 of Table 4, or a salt thereof. In some embodiments, the A3AR agonist is not IV-7 of Table 4, or a salt thereof. In some embodiments, the A3AR agonist is not IV-8 of Table 4, or a salt thereof. In some embodiments, the A3AR agonist is not IV-9 of Table 4, or a salt thereof. In some embodiments, the A3AR agonist is not IV-10 of Table 4, or a salt thereof. In some embodiments, the A3AR agonist is not IV-11 of Table 4, or a salt thereof. In some embodiments, the A3AR agonist is not IV-12 of Table 4, or a salt thereof. In some embodiments, the A3AR agonist is not IV-13 of Table 4, or a salt thereof. In some embodiments, the A3AR agonist is not IV-14 of Table 4, or a salt thereof. In some embodiments, the A3AR agonist is not IV-15 of Table 4, or a salt thereof. In some embodiments, the A3AR agonist is not IV-16 of Table 4, or a salt thereof. In some embodiments, the A3AR agonist is not IV-17 of Table 4, or a salt thereof. In some embodiments, the A3 AR agonist is not IV-48 of Table 4, or a salt thereof. In some embodiments, the A3 AR agonist is not IV-19 of Table 4, or a salt thereof. In some embodiments, the A3AR agonist is not IV-20 of Table 4, or a salt thereof. In some embodiments, the A3AR agonist is not IV-21 of Table 4, or a salt thereof. In some embodiments, the A3AR agonist is not IV-22 of Table 4, or a salt thereof. In some embodiments, the A3AR agonist is not IV-23 of Table 4, or a salt thereof. In some embodiments, the A3AR agonist is not IV-24 of Table 4, or a salt thereof. In some embodiments, the A3AR agonist is not IV-25 of Table 4, or a salt thereof. In some embodiments, the A3AR agonist is not IV-26 of Table 4, or a salt thereof. In some embodiments, the A3AR agonist is not IV-27 of Table 4, or a salt thereof. In some embodiments, the A3AR agonist is not IV-28 of Table 4, or a salt thereof. In some embodiments, the A3AR agonist is not IV-29 of Table 4, or a salt thereof. In some embodiments, the A3AR agonist is not IV-30 of Table 4, or a salt thereof. In some embodiments, the A3 AR agonist is not IV-31 of Table 4, or a salt thereof. In some embodiments, the A3 AR agonist is not IV-31 of Table 4, or a salt thereof. In some embodiments, the A3 AR agonist is not IV-33 of Table 4, or a salt thereof.

In some embodiments, the A3AR agonist is a compound listed in Table 4, or a salt thereof, or any combination of the compounds or salt thereof listed in Table 4. In some embodiments, the A3AR agonist is IV-1 of Table 4, or a salt thereof. In some embodiments, the A3 AR agonist is IV-2 of Table 4, or a salt thereof. In some embodiments, the A3 AR agonist is IV-3 of Table 4, or a salt thereof. In some embodiments, the A3AR agonist is IV-4 of Table 4, or a salt thereof. In some embodiments, the A3AR agonist is IV-5 of Table 4, or a salt thereof. In some embodiments, the A3 AR agonist is IV-6 of Table 4, or a salt thereof. In some embodiments, the A3AR agonist is IV-7 of Table 4, or a salt thereof. In some embodiments, the A3 AR agonist is IV-8 of Table 4, or a salt thereof. In some embodiments, the A3 AR agonist is IV-9 of Table 4, or a salt thereof. In some embodiments, the A3AR agonist is IV-10 of Table 4, or a salt thereof. In some embodiments, the A3AR agonist is IV-11 of Table 4, or a salt thereof. In some embodiments, the A3AR agonist is IV-12 of Table 4, or a salt thereof. In some embodiments, the A3AR agonist is IV-13 of Table 4, or a salt thereof. In some embodiments, the A3AR agonist is IV-14 of Table 4, or a salt thereof. In some embodiments, the A3AR agonist is IV-15 of Table 4, or a salt thereof. In some embodiments, the A3AR agonist is IV-16 of Table 4, or a salt thereof. In some embodiments, the A3AR agonist is IV-17 of Table 4, or a salt thereof. In some embodiments, the A3AR agonist is IV-18 of Table 4, or a salt thereof. In some embodiments, the A3AR agonist is IV-19 of Table 4, or a salt thereof. In some embodiments, the A3AR agonist is IV-20 of Table 4, or a salt thereof. In some embodiments, the A3AR agonist is IV-21 of Table 4, or a salt thereof. In some embodiments, the A3AR agonist is IV-22 of Table 4, or a salt thereof. In some embodiments, the A3AR agonist is IV-23 of Table 4, or a salt thereof. In some embodiments, the A3AR agonist is IV-24 of Table 4, or a salt thereof. In some embodiments, the A3AR agonist is IV-25 of Table 4, or a salt thereof. In some embodiments, the A3AR agonist is IV-26 of Table 4, or a salt thereof. In some embodiments, the A3AR agonist is IV-27 of Table 4, or a salt thereof. In some embodiments, the A3AR agonist is IV-28 of Table 4, or a salt thereof. In some embodiments, the A3AR agonist is IV-29 of Table 4, or a salt thereof. In some embodiments, the A3AR agonist is IV-30 of Table 4, or a salt thereof. In some embodiments, the A3AR agonist is IV-31 of Table 4, or a salt thereof. In some embodiments, the A3AR agonist is IV-31 of Table 4, or a salt thereof. In some embodiments, the A3AR agonist is IV-33 of Table 4, or a salt thereof.

In some embodiments, the A3 AR agonist is selected from

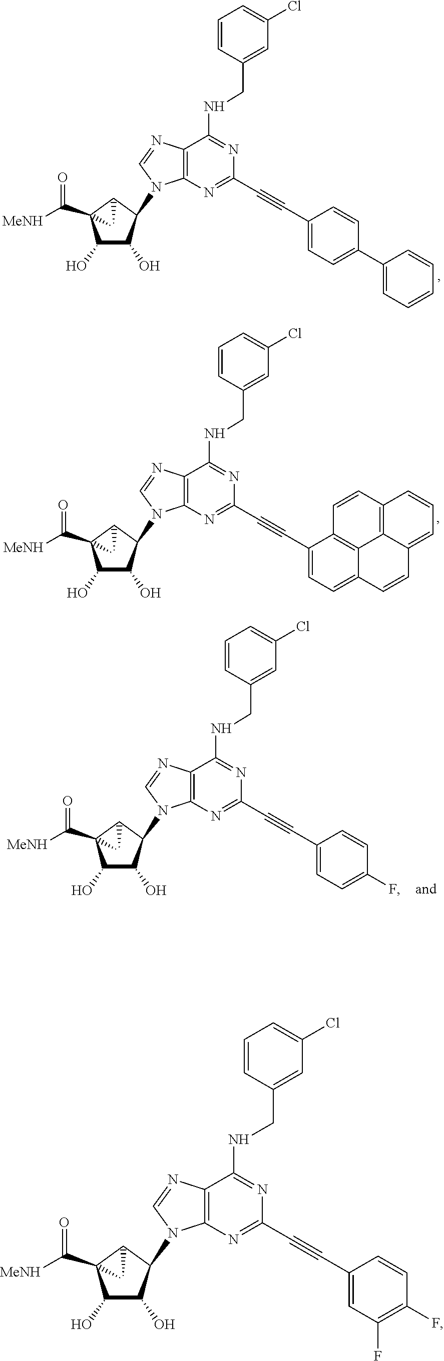

and a a salt of any one thereof. In some embodiments, the A3AR agonist is

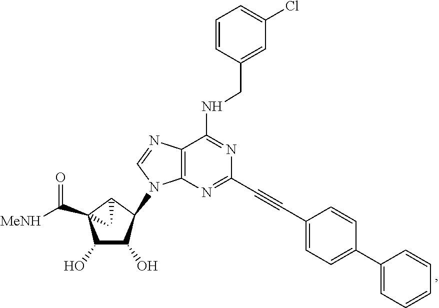

or a salt thereof. In some embodiments, the A3AR agonist is

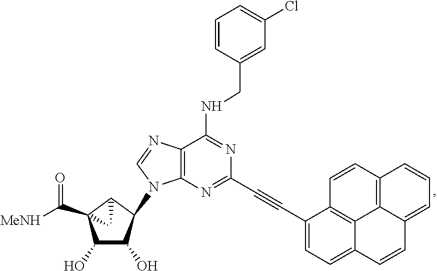

or a salt thereof. In some embodiments, the A3AR agonist is

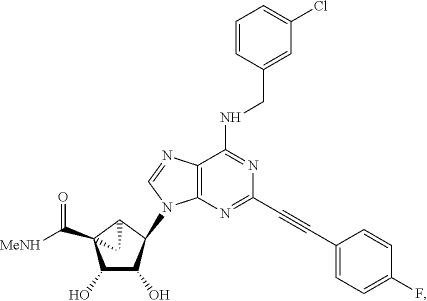

or a salt thereof. In some embodiments, the A3AR agonist is

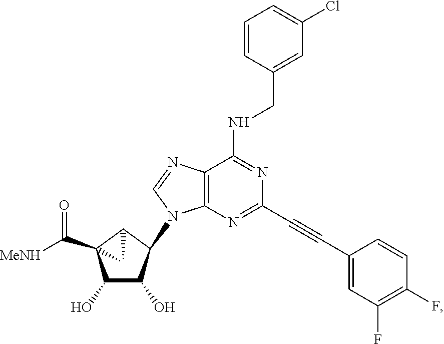

or a salt thereof.

In certain embodiments, the selective agonist for the human adenosine A3 receptor subtype (A3AR agonist) is a compound of Formula (G):

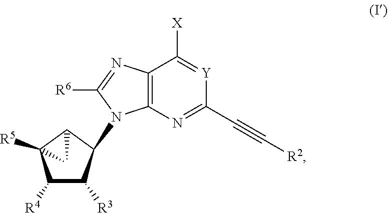

or a pharmaceutically acceptable salt thereof, wherein:
X is selected from NR^{o}R′, CFb, and CH═C(R^{a})(R^{b}),
wherein R^{a} and R^{b} are independently selected from hydrogen, hydroxyl, C₁-C₆ alkyl, and C₆-C14 aryl;

### Y is N or CH;

R^{o} is hydrogen or CH₃;
R¹ is selected from hydrogen, C₁-C₆ alkyl, C₁-C₆ alkoxy, hydroxyl, C₃-C₈ cycloalkyl, C₃-Ca cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci₄ aryl C₁-C₆ alkyl, C6-C14 diaryl C1-C6 alkyl, C6-Ci4 aryl C1-C6 alkoxy, heterocyclyl C1-C6 alkyl, heterocyclyl, 4-[[[4-[[[(2-amino C₁-C₆ alkyl) amino]-carbonyl]-C₁-C₆ alkyl] anilino] carbonyl] C₁-C₆ alkyl] C₆-Ci₄ aryl, and C₆-Ci₄ aryl C₃-C₈ cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C6 alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and arylalkoxy;
R² is selected from C₆-C₁₂ aryl, C₃-C₈ cycloalkyl, heteroaryl, and metallocenyl, wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl;
R³ and R⁴ are independently selected from hydrogen, hydroxyl, amino, mercapto, ureido, C₁-C₆ alkyl carbonylamino, hydroxy C₁-C₆ alkyl, and hydrazinyl;
R⁵ is selected from C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-Ce cycloalkylamino, hydroxylamino, and C₂-C₃ alkenyl; and
R⁶ is selected from hydrogen, C₁-C₆ alkyl, C₂-C₆ alkenyl, C₂-C₆ alkynyl, heteroaryl, and Ci-C₆ aminoalkyl.

In certain embodiments for a compound of formula (I), wherein when R¹ is halobenzyl or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylmethyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

In certain embodiments, the selective agonist for the human adenosine A3 receptor subtype (A3AR agonist) is a compound of Formula (G):

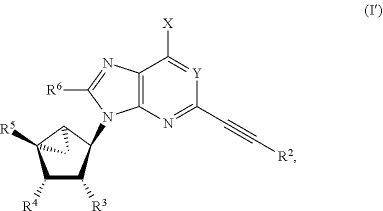

or a pharmaceutically acceptable salt thereof, wherein:
X is selected from NR^{o}R′, CH₃, and CH═C(R^{a})(R^{b}),
wherein R^{a} and R^{b} are independently selected from hydrogen, hydroxyl, C₁-C₆ alkyl, and C₆-C14 aryl;

### Y is N or CH;

R^{o} is hydrogen or CfR;
R¹ is selected from hydrogen, C₁-C₆ alkyl, C₁-C₆ alkoxy, hydroxyl, C₃-C₈ cycloalkyl, C₃-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci₄ aryl C₁-C₆ alkyl, C6-C14 diaryl C1-C6 alkyl, C6-Ci4 aryl C1-C6 alkoxy, heterocyclyl C1-C6 alkyl, heterocyclyl, and C₆-C14 aryl C3-C8 cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C6 alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;
R² is selected from C₆-C₁₂ aryl, C₃-C₈ cycloalkyl, heteroaryl, and metallocenyl, wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl;
R³ and R⁴ are independently selected from hydrogen, hydroxyl, amino, mercapto, ureido, C₁-C₆ alkyl carbonylamino, hydroxy C₁-C₆ alkyl, and hydrazinyl;
R⁵ is selected from C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C₆ cycloalkylamino, hydroxylamino, and C₂-C₃ alkenyl; and
R⁶ is selected from hydrogen, C₁-C₆ alkyl, C₂-C₆ alkenyl, C₂-C₆ alkynyl, heteroaryl, and C₁-C₆ aminoalkyl, with the proviso that:
when R¹ is methyl, then R² is selected from the group consisting of

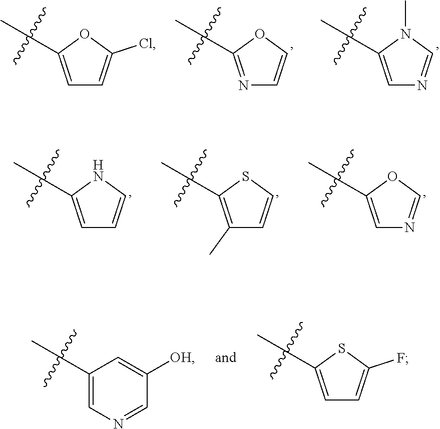

and
when R¹ is halobenzyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

In certain embodiments, the A3AR agonist is a compound of the Formula (G) is a compound of Formula (I):

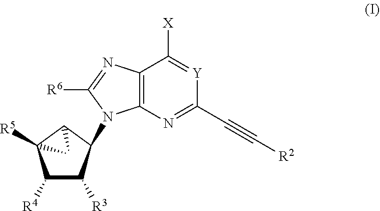

wherein: X is selected from NHR¹, CH₃, and CH═C(R^{a})(R^{b}) wherein R^{a} and R^{b} are independently selected from hydrogen, hydroxyl, C₁-C₆ alkyl, and C₆-C₁₄ aryl;

Y is N or CH;

R¹ is selected from C₁-C₆ alkyl, C₁-C₆ alkoxy, hydroxyl, C₃-C₈ cycloalkyl, C₆-C₁₄ aryl C₃-C₈ cycloalkyl, C₃-C₈ cycloalkyl C₁-C₆ alkyl, C₃-C₈ dicycloalkyl C₁-C₆ alkyl, C₇-Ci₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci4 aryl C1-C6 alkyl, C6-C14 diaryl C1-C6 alkyl, C6-C14 aryl C1-C6 alkoxy, heterocyclyl C1-C6 alkyl, heterocyclyl, 4-[[[4-[[[(2-amino C₁-C₆ alkyl) amino]-carbonyl]-C₁-C₆ alkyl] anilino] carbonyl] C₁-C₆ alkyl] C₆-C₁₄ aryl, and C₆-C₁₄ aryl C₃-C₈ cycloalkyl, wherein the aryl or heterocyclyl portion of R¹ is optionally substituted with one or more substituents selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, C₁-C₆ alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, aminocarbonyl C₂-C₆ alkynyl, and any combination thereof; and the alkyl or cycloalkyl portion of R¹ is optionally substituted with one or more substituents selected from halo, amino, alkyl, alkoxy, aryloxy, hydroxyalkyl, hydroxyalkenyl, hydroxyalkynyl, aminocarbonylalkoxy, and arylalkoxy, and any combination thereof;

R² is selected from C₆-C₁₂ aryl, C₃-C₈ cycloalkyl, heteroaryl, and metallocenyl, wherein the aryl group is substituted with one or more substituents selected from trifluoromethyl, hydroxyalkyl, alkoxy, sulfonyloxy, carboxyalkyl, sulfonyloxyalkyl, arylcarbonyl, and any combination thereof, wherein the heteroaryl group is optionally substituted with one or more substituents selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, arylcarbonyl, and any combination thereof;

R³ and R⁴ are independently selected from hydrogen, hydroxyl, amino, mercapto, ureido, C₁-C₆ alkyl carbonylamino, hydroxy C₁-C₆ alkyl, and hydrazinyl;

R⁵ is selected from C₁-C₃ alkyl aminocarbonyl, di(Ci-C₃ alkyl) aminocarbonyl, C1-C3 alkylthio C1-C3 alkyl, halo C1-C3 alkyl, hydrazinyl, amino C1-C3 alkyl, hydroxy C1-C3 alkyl, C₃-C₆ cycloalkylamino, hydroxylamino, and C₂-C₃ alkenyl; and

R⁶ is selected from hydrogen, C₁-C₆ alkyl, C₂-C₆ alkenyl, C₂-C₆ alkynyl, heteroaryl, and C₁-C₆ aminoalkyl;

or a pharmaceutically acceptable salt thereof.

In certain embodiments for a compound of formula (I), X is NHR¹, R¹ is C₁-C₆ alkyl, R² is C₆-C₁₀ aryl, wherein the aryl group is substituted with one or more substituents selected from halo, trifluoromethyl, hydroxyalkyl, alkoxy, and any combination thereof, or R² is heteroaryl, and the heteroaryl group is optionally substituted with one or more substituents selected from halo, hydroxy, and alkyl.

In certain embodiments for a compound of formula (I), when R¹ is methyl, R³ and R⁴ are both hydroxyl, R⁶ is hydrogen, and R⁵ is methylaminocarbonyl, R² is not 2-pyridyl or phenyl.

In certain embodiments for a compound of formula (I), when R¹ is methyl, R³ and R⁴ are both hydroxyl, R⁶ is hydrogen, and R is methylaminocarbonyl, R² is not 2-pyridyl.

In certain embodiments for a compound of formula (I), R⁶ is hydrogen.

In certain embodiments for a compound of formula (I), Y is N.

In certain embodiments for a compound of formula (I), R⁵ is selected from C₁-C₃ alkyl aminocarbonyl or di(Ci-C₃ alkyl) aminocarbonyl.

In certain embodiments for a compound of formula (I), R³ and R⁴ are both hydroxyl.

In certain embodiments for a compound of formula (I), X is NHR¹. In a preferred embodiment, R¹ is C₁-C₆ alkyl.

In certain embodiments for a compound of formula (I), R² is C6-C10 aryl, wherein the aryl group is substituted with one or more substituents selected from halo, trifluoromethyl, hydroxyalkyl, alkoxy, and any combination thereof.

In certain embodiments for a compound of formula (I), R² is heteroaryl, and the heteroaryl group is optionally substituted with one or more substituents selected from hydroxy, halo and alkyl.

In certain embodiments for a compound of formula (I), R¹ is selected from C₁-C₆ alkyl, C₁-C₆ alkoxy, hydroxyl, C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-Ci₄ tricycloalkyl C₁-C₆ alkyl, C6-C14 aryl, C6-C14 aryl C1-C6 alkyl, C6-C14 diaryl C1-C6 alkyl, C6-C14 aryl C1-C6 alkoxy, heterocyclyl C₁-C₆ alkyl, heterocyclyl, and C₆-Ci₄ aryl C₃-C₈ cycloalkyl,

wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl Ci-Ce alkyl, aminocarbonyl C₂-C6 alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and arylalkoxy.

In certain embodiments for a compound of formula (I), when R¹ is methyl, then R is selected from the group consisting of

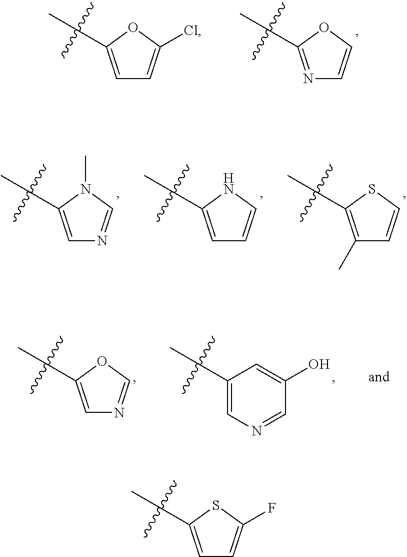

In certain embodiments for a compound of formula (I),
when R¹ is methyl, then R² is selected from the group consisting of

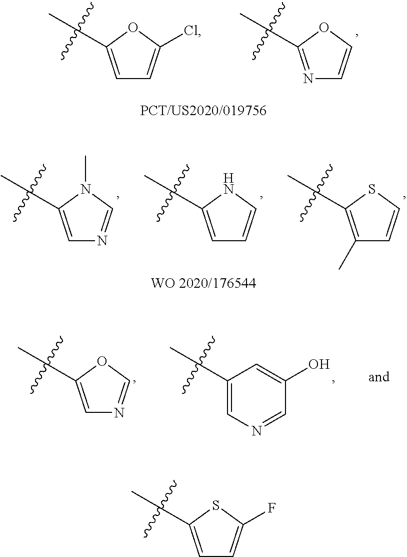

In certain embodiments for a compound of formula (I), when R¹ is methyl, then R² is

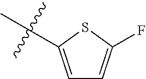 [SMILES: *c1ccc(F)s1]

In certain embodiments for a compound of formula (I), when R¹ is methyl, then R² is

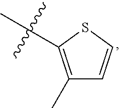 [SMILES: *c1sccc1C]

or V′-VJ^{F}. In certain embodiments for a compound of formula (I), when R¹ is methyl, then R² is selected from the group consisting of

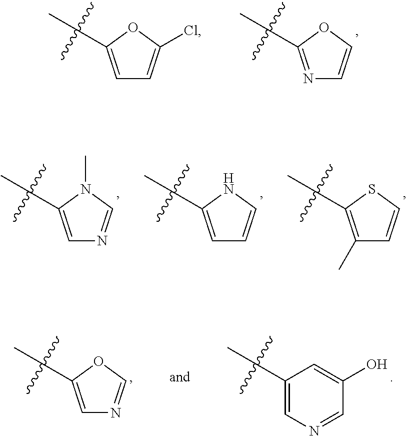

In certain embodiments for a compound of formula (I), wherein when R¹ is halobenzyl or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylethyl, or phenylethyl, then R² is selected from the group consisting of C₁₀-C₁₂ aryl, C₃-C cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), wherein when R¹ is halobenzyl, diphenylmethyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), when R¹ is halobenzyl, diphenylmethyl, diphenylethyl, or phenylethyl, then R² is not an optionally substituted phenyl. In certain embodiments for a compound of formula (I), when R¹ is halobenzyl, diphenylmethyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), when R¹ is halobenzyl, diphenylmethyl, diphenylethyl, or phenylethyl, then R² is thienyl, wherein the thienyl group of R² is optionally substituted with one or more halo, each independently selected.

In certain embodiments for a compound of formula (I), when R¹ is halobenzyl, diphenylmethyl, diphenyl ethyl, or phenyl ethyl, then R² is halothienyl. In certain embodiments for a compound of formula (I), when R¹ is benzyl, halobenzyl, diphenylmethyl, diphenylethyl, or phenylethyl, then R² is not an optionally substituted phenyl. In certain embodiments for a compound of formula (I), when R¹ is benzyl, halobenzyl, diphenylmethyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected. In certain embodiments for a compound of formula (I), when R¹ is benzyl, halobenzyl, diphenylmethyl, diphenylethyl, or phenylethyl, then R² is thienyl, wherein the thienyl group of R² is optionally substituted with one or more halo, each independently selected. In certain embodiments for a compound of formula (I), when R¹ is benzyl, halobenzyl, diphenylmethyl, diphenylethyl, or phenylethyl, then R² is halothienyl.

In certain embodiments for a compound of formula (I), when R¹ is an optionally substituted C₆ aryl C₁-C₂ alkyl or optionally substituted di-C₆ aryl C₁-C₂ alkyl, then R² is not an optionally substituted phenyl. In certain embodiments for a compound of formula (I), when R¹ is an optionally substituted C₆ aryl C₁-C₂ alkyl or optionally substituted di-C₆ aryl C₁-C₂ alkyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

In certain embodiments for a compound of formula (I), when R¹ is an optionally substituted C₆ aryl C₁-C₂ alkyl or optionally substituted di-C₆ aryl C₁-C₂ alkyl, then R² is an optionally substituted thienyl. In certain embodiments for a compound of formula (I), when R¹ is an optionally substituted C₆ aryl C₁-C₂ alkyl or optionally substituted di-C₆ aryl C₁-C₂ alkyl, then R² is halothienyl.

In certain embodiments for a compound of formula (I), X is selected from CH₃, CH═C(R^{a})(R^{b}), and NHR¹, wherein:

R¹ is selected from C₂-C₆ alkyl, Ci-C₆ alkoxy, hydroxyl, C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl Ci-C₆ alkoxy, heterocyclyl C₁-C₆ alkyl, heterocyclyl, and C₆-C₁₄ aryl C₃-C₈ cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, Ci-C₆ alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C2-C6 alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and arylalkoxy.

In some embodiments for a compound of formula (I), R¹ is selected from C2-C6 alkyl, C3-C8 cycloalkyl C1-C6 alkyl and C3-C8 cycloalkyl, wherein the alkyl or cycloalkyl portion of R¹ is optionally substituted with one or more substituents each independently selected from halo, amino, C1-C6 alkyl, C1-C6 alkoxy, C6-C10 aryloxy, hydroxy(Ci-C₆)alkyl, hydroxy(C2-Ce)alkenyl, hydroxy(C2-Ce)alkynyl, aminocarbonyl(Ci-C₆)alkoxy, and C6-C10 arylalkoxy.

In certain embodiments for a compound of formula (I), when R¹ is halobenzyl, diphenylmethyl, diphenylethyl, or phenylethyl, then R² is an optionally substituted thienyl. In certain embodiments for a compound of formula (I), when R¹ is halobenzyl, diphenylmethyl, diphenylethyl, or phenylethyl, then R² is thienyl, wherein the thienyl group of R² is optionally substituted with one or more halo, each independently selected.

In certain embodiments for a compound of formula (I), R¹ is selected from C2-C6 alkyl, C₃-C₈ cycloalkyl C₁-C₆ alkyl and C₃-C₈ cycloalkyl.

In certain embodiments for a compound of formula (I), R¹ is selected from C₂-C₃ alkyl, C₃-C₄ cycloalkyl, and C₃-C₄ cycloalkyl C₁-C₃ alkyl.

In certain embodiments for a compound of formula (I), R¹ is selected from ethyl, n-propyl, i-propyl, n-butyl, cylcopropyl, cyclobutyl, cylcopropylmethyl, and cyclobutylmethyl.

In certain embodiments for a compound of formula (I), when R¹ is C₂-C₃ alkyl, then R² is selected from C₆-C₁₂ aryl, C₃-C₈ cycloalkyl, heteroaryl, and metallocenyl, wherein the aryl group of R² is substituted with one or more substituents each independently selected from trifluoromethyl, hydroxyalkyl, alkoxy, sulfonyloxy, carboxyalkyl, sulfonyloxyalkyl, and arylcarbonyl; and wherein the heteroaryl group of R² is optionally substituted with one or more substituents each independently selected from fluoro, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl.

In certain embodiments for a compound of formula (I), when R¹ is C₂-C₃ alkyl, then R² is selected from C₆-C₁₂ aryl, C₃-C₈ cycloalkyl, metallocenyl, methylthienyl, fluorothienyl, and 5 or 6 membered heteroaryl comprising one or more heteroatoms independently selected from N and O, wherein the aryl group of R² is substituted with one or more substituents each independently selected from trifluoromethyl, hydroxyalkyl, alkoxy, sulfonyloxy, carboxyalkyl, sulfonyloxyalkyl, and arylcarbonyl; and wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl.

In certain embodiments for a compound of formula (I), when R¹ is C2-C3 alkyl, then R² is selected from methyl thienyl, fluorothienyl, and 5 or 6 membered heteroaryl comprising one or more heteroatoms are each independently selected from N and O, wherein the 5 or 6 membered heteroaryl group of R² is optionally substituted with one or more substituents each independently selected from halo, C1-C3 alkyl, and hydroxyl.

In certain embodiments for a compound of formula (I), the 5 or 6 membered heteroaryl comprising one or more heteroatoms each independently selected from N and O is selected from furanyl, oxazolyl, imidazolyl, pyrrolyl, pyridyl, and pyrazinyl.

In certain embodiments for a compound of formula (I), when R¹ is methyl, R³ and R⁴ are both hydroxyl, R⁶ is hydrogen, and R⁵ is methylaminocarbonyl, R² is not 2-pyridyl or phenyl.

In certain embodiments for a compound of formula (I), when R¹ is methyl, R³ and R⁴ are both hydroxyl, R⁶ is hydrogen, and R⁵ is methylaminocarbonyl, R² is not 2-pyridyl.

In certain embodiments for a compound of formula (I), R⁶ is hydrogen.

In certain embodiments for a compound of formula (I), Y is N.

In certain embodiments for a compound of formula (I), R⁵ is selected from C1-C3 alkyl aminocarbonyl or di(Ci-C₃ alkyl) aminocarbonyl.

In certain embodiments for a compound of formula (I), R⁵ is methylaminocarbonyl.

In certain embodiments for a compound of formula (I), R³ is hydroxyl.

In certain embodiments for a compound of formula (I), R⁴ is hydroxyl.

In certain embodiments for a compound of formula (I), R³ and R⁴ are both hydroxyl.

In certain embodiments for a compound of formula (I), R² is C6-C10 aryl, wherein the aryl group of R² is substituted with one or more substituents each independently selected from halo, trifluoromethyl, hydroxyalkyl, and alkoxy.

In certain embodiments for a compound of formula (I), R² is a 5 or 6 membered heteroaryl, and the heteroaryl group of R² is optionally substituted with one or more substituents each independently selected from hydroxy, halo and C1-C6 alkyl.

In certain embodiments for a compound of formula (I), R² is thienyl, furanyl, or pyrazinyl, wherein the thienyl, furanyl, or pyrazinyl of R² is optionally substituted with one or more substituents each independently selected from hydroxy, halo, and C1-C6 alkyl.

In certain embodiments for a compound of formula (I), R² is selected from furanyl, halothienyl, and pyrazinyl.

In certain embodiments for a compound of formula (I), R² is selected from furanyl, fluorothienyl, and pyrazinyl.

In certain embodiments for a compound of formula (I), R² is selected from furanyl, fluorothienyl, and pyrazinyl.

In certain embodiments for a compound of formula (I), when X is NHR¹ and R¹ is C2-C3 alkyl, then R² is not 2-halothienyl.

In certain embodiments for a compound of formula (I), the compound is selected from:

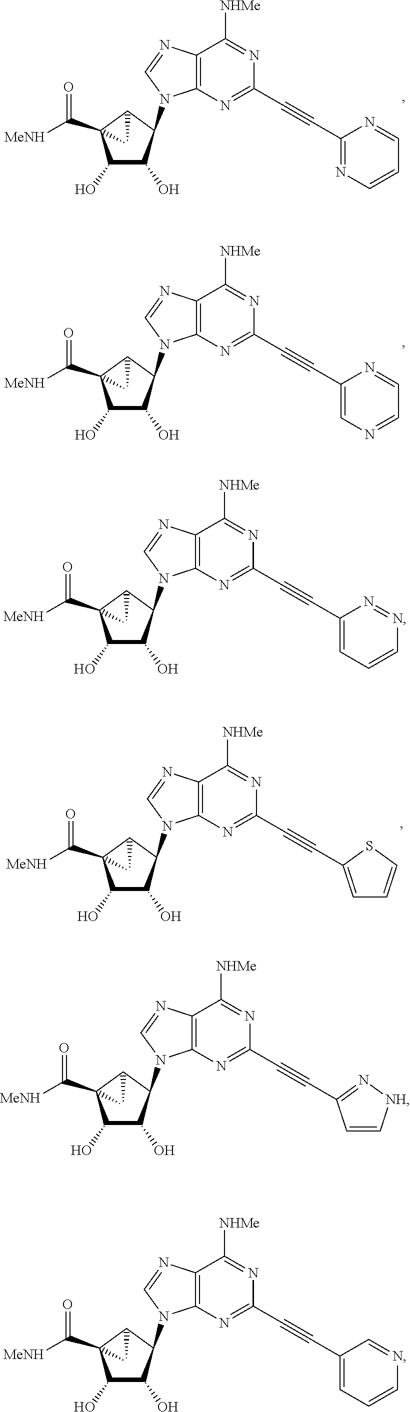

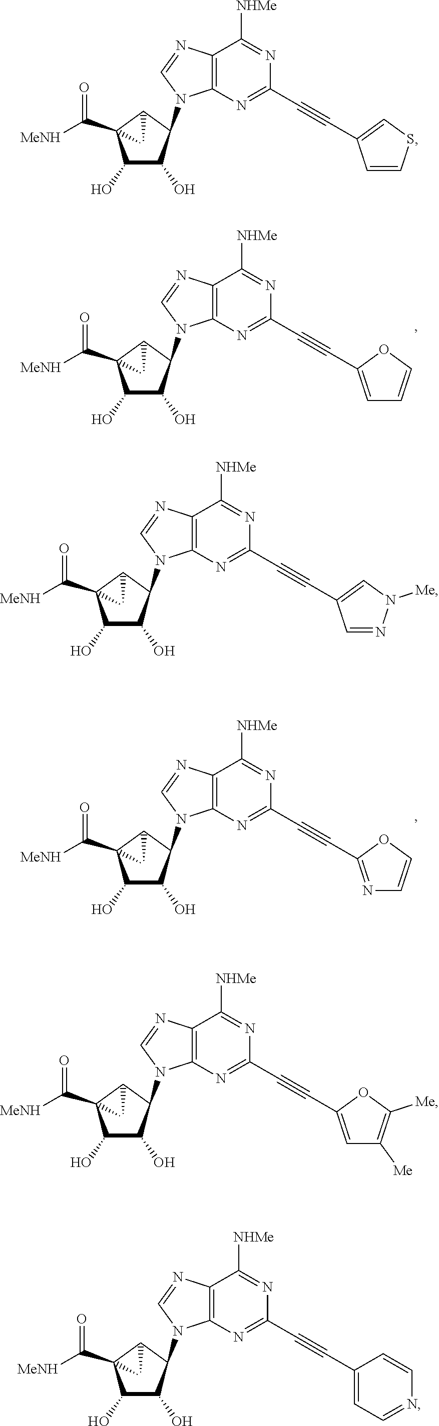

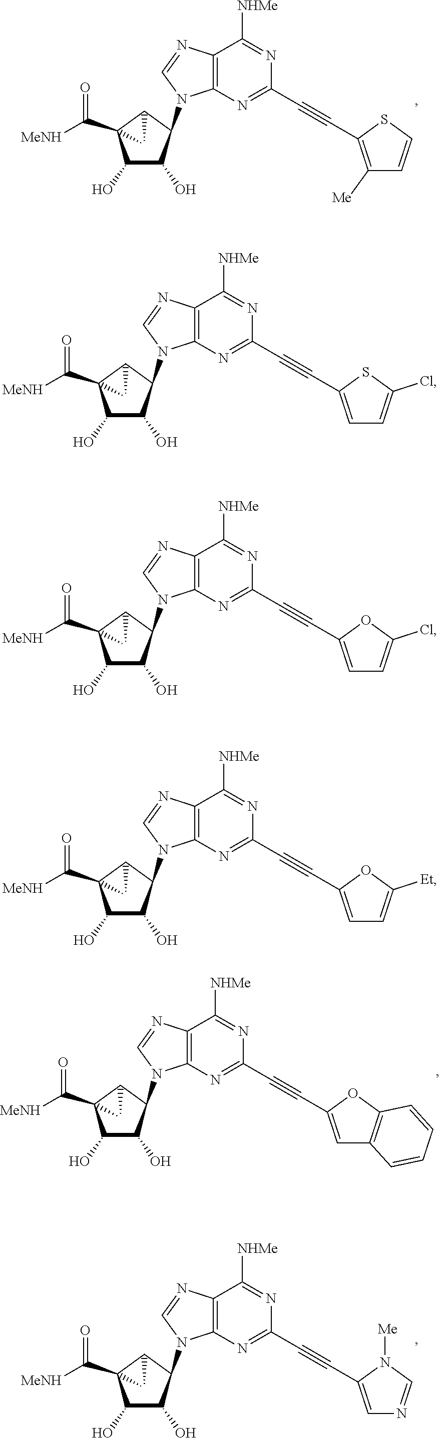

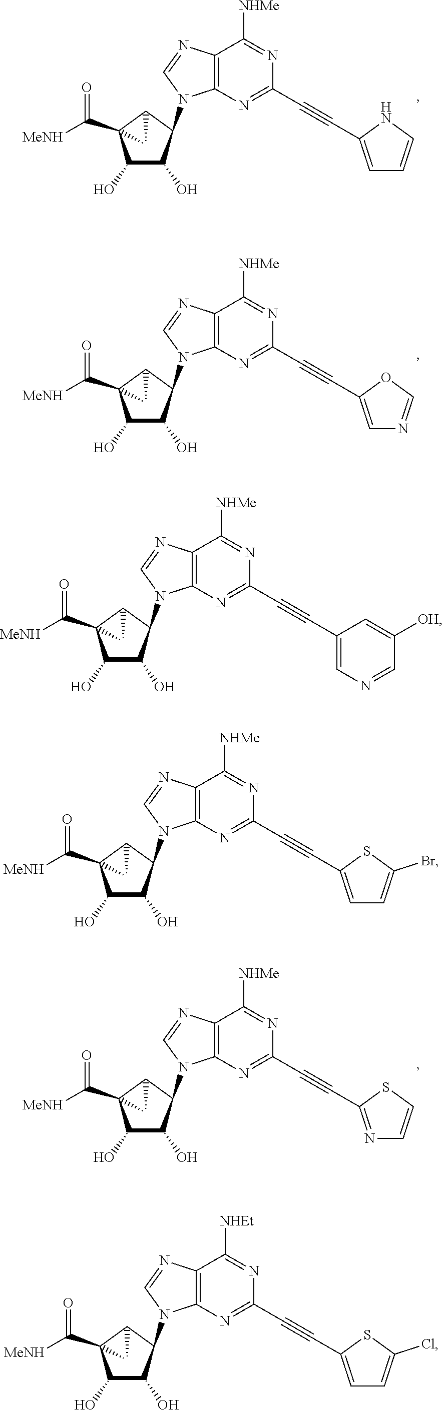

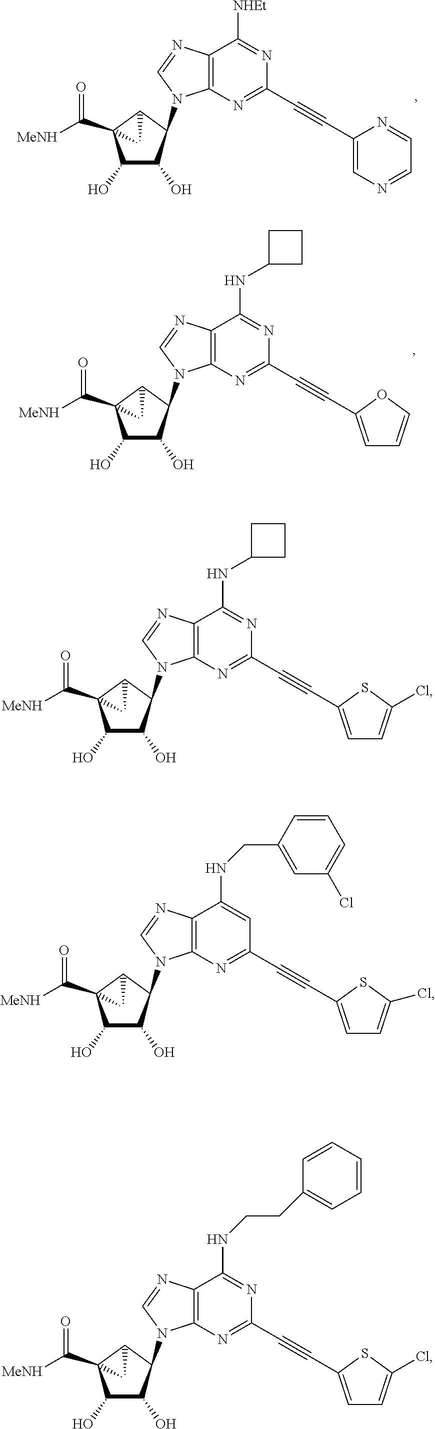

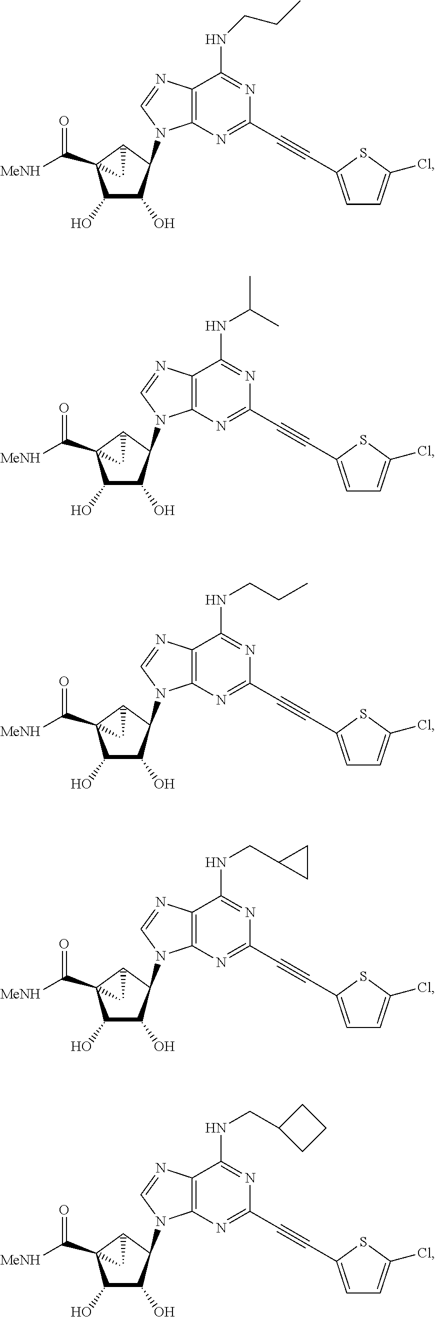

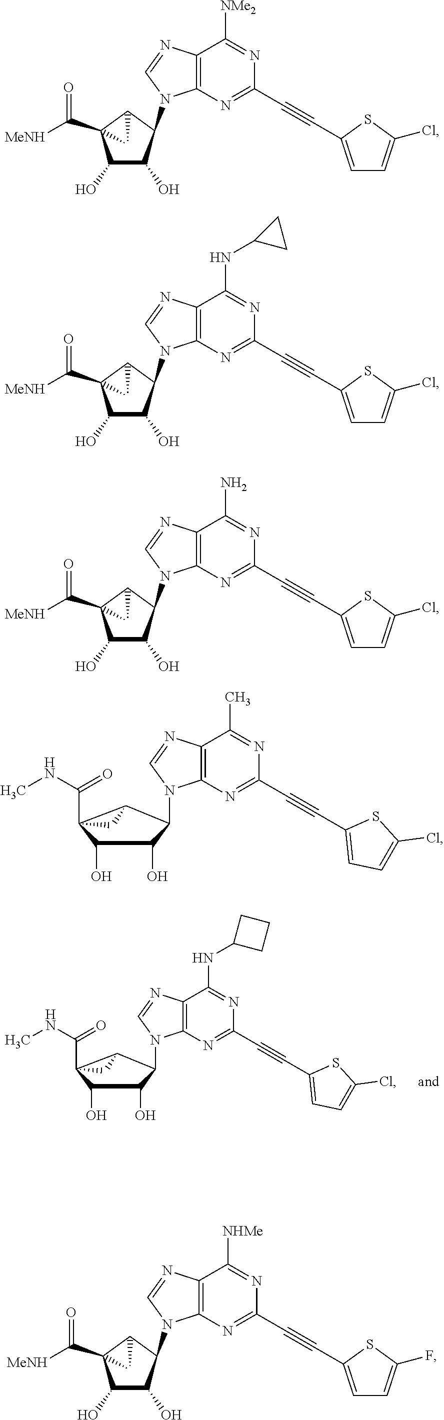

and a salt of any one thereof.

In certain embodiments for a compound of formula (I), the A3AR agonist is selected from:

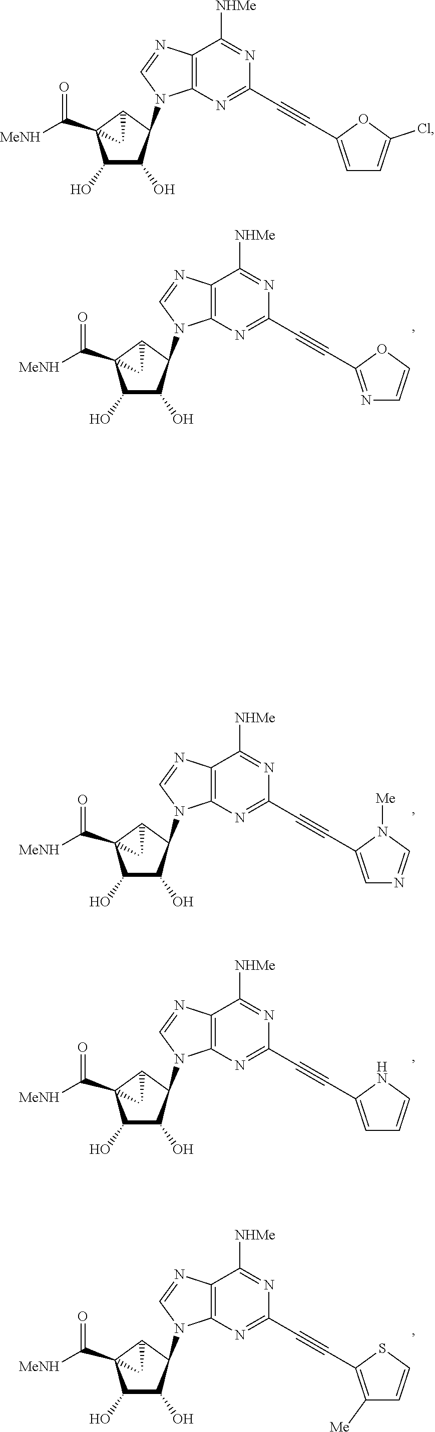

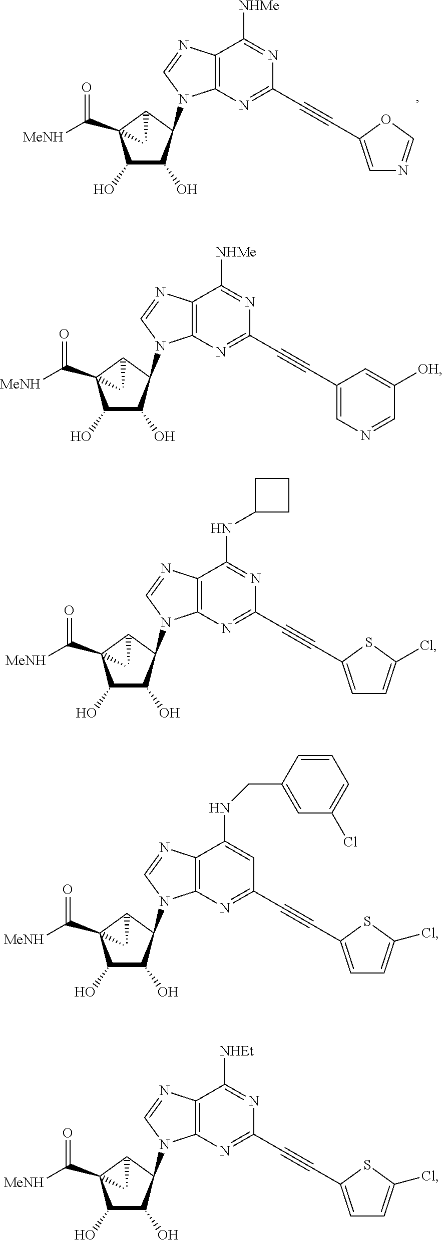

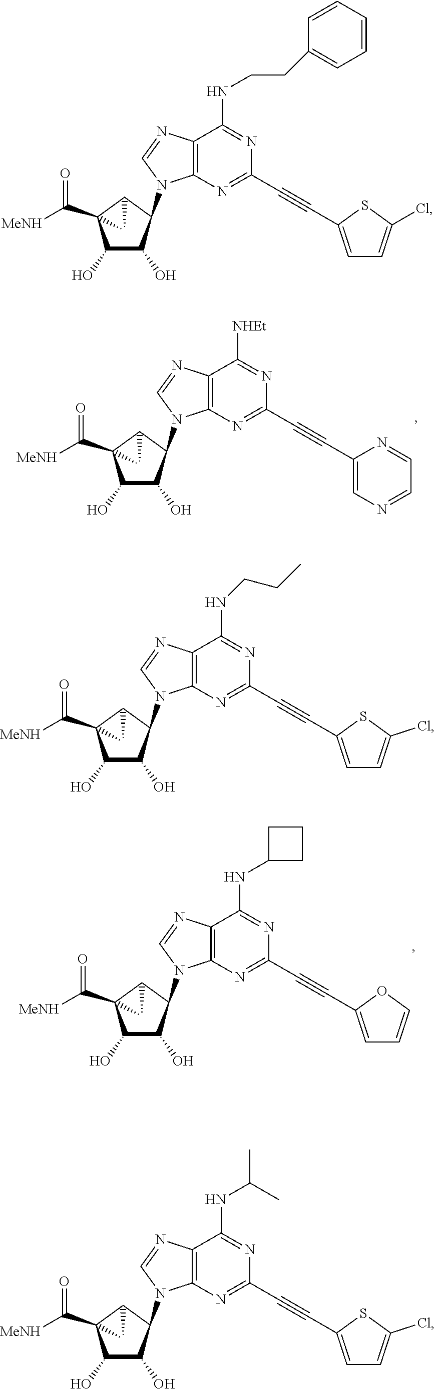

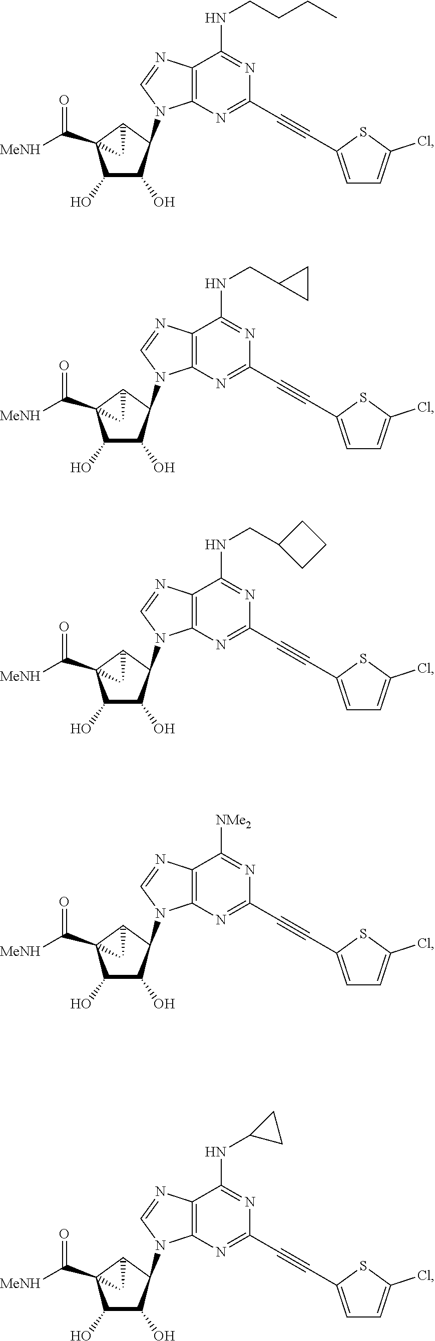

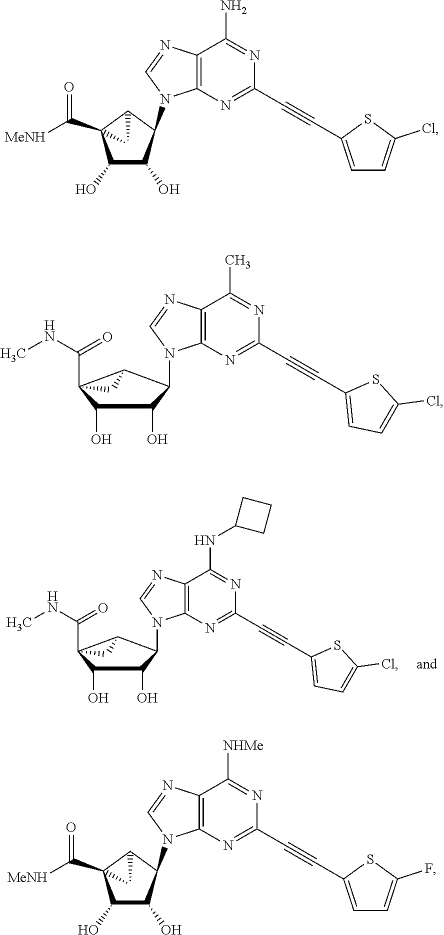

salt of any one thereof.

In certain embodiments for a compound of formula (I), the A3AR agonist is selected from

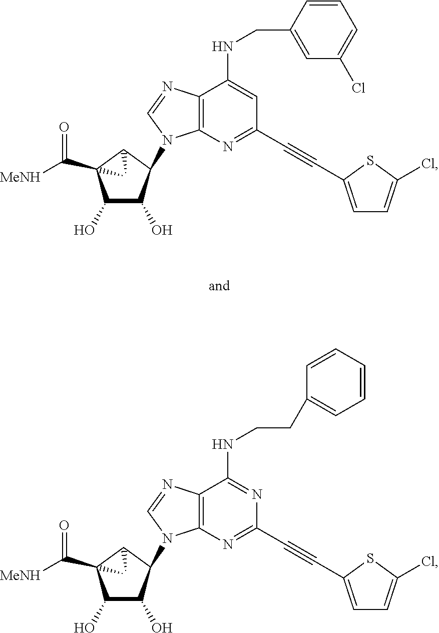

a salt of any one thereof.

In certain embodiments for a compound of formula (I), the A3AR agonist is selected from:

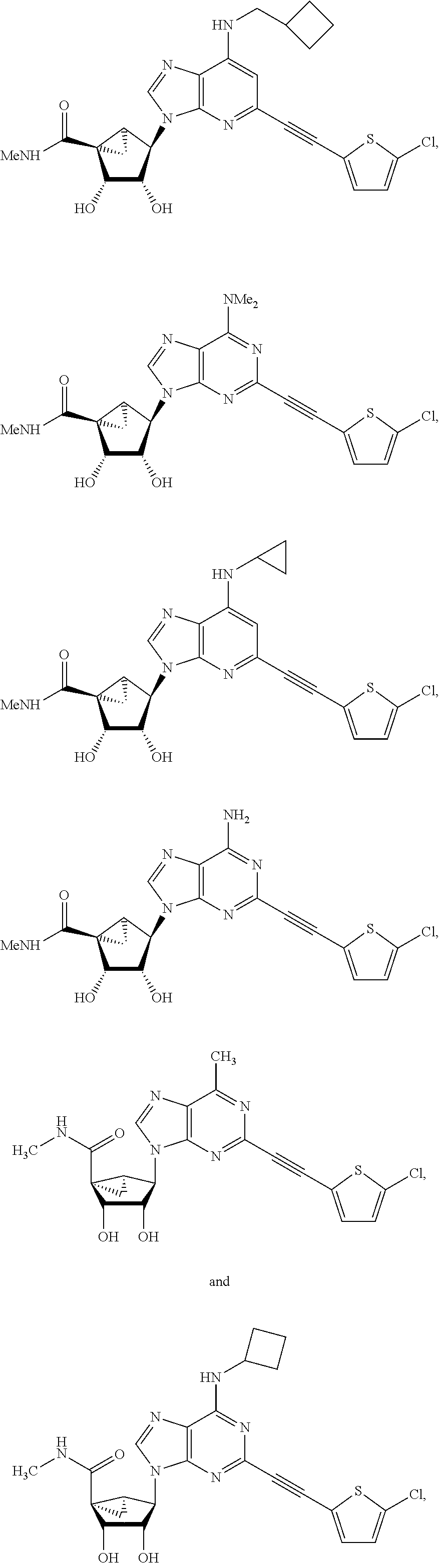

and a salt ofany one thereof.

In certain embodiments for a compound of formula (I),

the A3AR agonist is selected from:

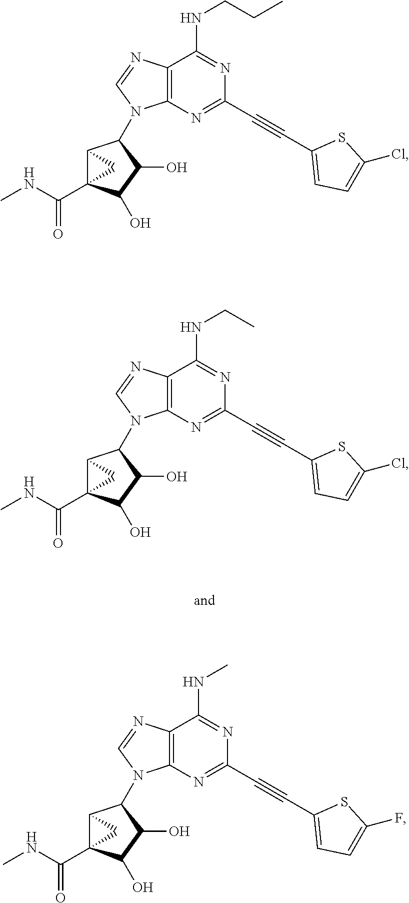

and a salt of any one thereof.

In certain embodiments for a compound of formula (I), the A3AR agonist is

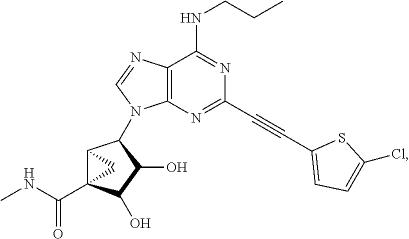

or a salt thereof.

In certain embodiments for a compound of formula (I), the A3AR agonist is

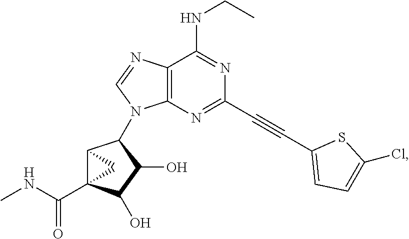

or a salt thereof.

In certain embodiments for a compound of formula (I), the A3AR agonist is

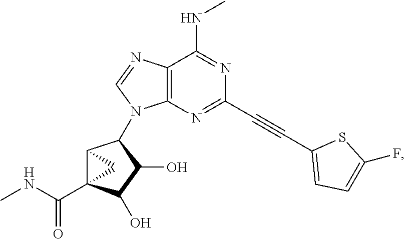

or a salt thereof.

In certain embodiments for a compound of formula (I), the A3AR agonist is

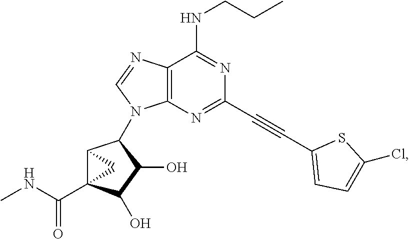

or a salt thereof.

In certain embodiments for a compound of formula (I), the A3AR agonist is

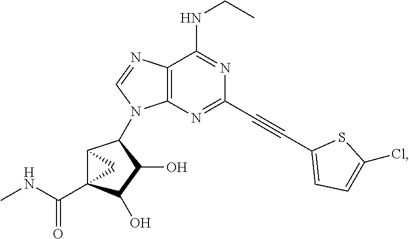

or a salt thereof.

In certain embodiments for a compound of formula (I), the A3AR agonist is

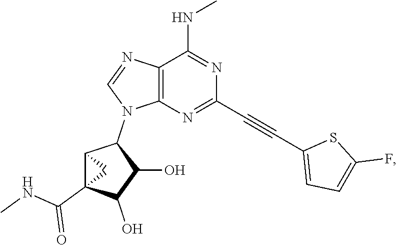

or a salt thereof.

In certain embodiments, the selective agonist for the adenosine A3 human receptor subtype (A3AR agonist) is a compound of Formula (IF):

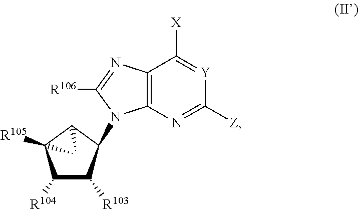

or a pharmaceutically acceptable salt thereof, wherein:
X is selected from NHR¹⁰¹, CH₃, and CH═C(R^{a})(R^{b}),
wherein R^{a} and R^{b} are independently selected from hydrogen, hydroxyl, C₁-C₆ alkyl, and C₆-C14 aryl;

### Y is N or CH;

R¹⁰¹ is selected from C1-C₆ alkyl, C1-C₆ alkoxy, hydroxyl, C3-C8 cycloalkyl, C3-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl C₁-C₆ alkyl, C₆-C₁₄ diaryl C₁-C₆ alkyl, C₆-Ci₄ aryl C₁-C₆ alkoxy, heterocyclyl C₁-C₆ alkyl, heterocyclyl, 4-[[[4-[[[(2-amino C1-C₆ alkyl) amino]-carbonyl]-Ci-C6 alkyl] anilino] carbonyl] C1-C₆ alkyl] C₆-Ci4 aryl, and C6-Ci₄ aryl C₃-C₈ cycloalkyl,
wherein the aryl or heterocyclyl portion of R¹⁰¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C₂-C₆ alkynyl; and
wherein the alkyl or cycloalkyl portion of R¹⁰¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;

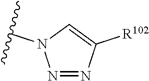

Z is halo, azido, or a group of the formula: ^{NN} wherein:
R¹⁰² is selected from Ce-Cn aryl, C₆-C₁₂ aryl-Ci-C6 alkyl, C₃-C₈ cycloalkyl, heteroaryl, and metallocenyl,
wherein the aryl or heteroaryl group of R¹⁰² is optionally substituted with one or more substituents each independently selected from halo, trifluoromethyl, amino, alkyl, hydroxyalkyl, aryl, alkoxy, hydroxyl, carboxyl, sulfonyloxy, carboxyalkyl, sulfonyloxyalkyl, alkylcarbonyl, and arylcarbonyl;
R¹⁰³ and R¹⁰⁴ are independently selected from hydrogen, hydroxyl, amino, mercapto, ureido, C₁-C₆ alkyl carbonylamino, hydroxy C₁-C₆ alkyl, and hydrazinyl;
R¹⁰⁵ is selected from hydrogen, C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C₆ cycloalkylamino, hydroxylamino, and C₂-C₃ alkenyl; and
R¹⁰⁶ is selected from hydrogen, C₁-C₆ alkyl, C₂-C₆ alkenyl, C₂-C₆ alkynyl, heteroaryl, and C₁-C₆ aminoalkyl.

In certain embodiments, the A3AR agonist is a compound of the formula (IG), with the proviso that, when R¹⁰³ and R¹⁰⁴ are both hydroxyl, R¹⁰⁵ is methylaminocarbonyl, R¹⁰⁶ is hydrogen, X is NHMe, and Y is CH, then Z is not iodo.

In certain embodiments, the A3AR agonist is a compound of the formula (IG) is a compound of Formula (II):

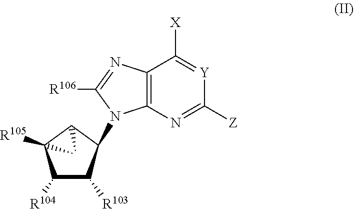

wherein: X is selected from NHR¹⁰¹, CH₃, and CH═C(R^{a})(R^{b}) wherein R^{a} and R^{b} are independently selected from hydrogen, hydroxyl, C₁-C₆ alkyl, and C₆-C₁₄ aryl;

Y is N or CH:

R¹⁰¹ is selected from C₁-C₆ alkyl, C₁-C₆ alkoxy, hydroxyl, C₃-C₈ cycloalkyl, C₆-Ci₄ aryl C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci4 aryl C1-C6 alkyl, C6-C14 diaryl C1-C6 alkyl, C6-C14 aryl C1-C6 alkoxy, heterocyclyl C1-C6 alkyl, heterocyclyl, 4-[[[4-[[[(2-amino Ci-C₆ alkyl) amino]-carbonyl]-C₁-C₆ alkyl] anilino] carbonyl] C₁-C₆ alkyl] C₆-C₁₄ aryl, and C₆-C₁₄ aryl C₃-C₈ cycloalkyl, wherein the aryl or heterocyclyl portion of R¹⁰¹ is optionally substituted with one or more substituents selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, C₁-C₆ alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, aminocarbonyl C₂-C₆ alkynyl, and any combination thereof; and the alkyl or cycloalkyl portion of R¹⁰¹ is optionally substituted with one or more substituents selected from halo, amino, alkyl, alkoxy, aryloxy, hydroxyalkyl, hydroxyalkenyl, hydroxyalkynyl, aminocarbonylalkoxy, and arylalkoxy, and any combination thereof;

Z is halo, azido, or a group of the formula:

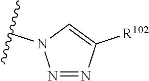

wherein R¹⁰² is selected from C6-C12 aryl, C6-C12 aryl-Ci-C6 alkyl, C3-C8 cycloalkyl, heteroaryl, and metallocenyl, wherein the aryl group is optionally substituted with one or more substituents selected from trifluoromethyl, hydroxyalkyl, alkoxy, sulfonyloxy, carboxyalkyl, sulfonyloxyalkyl, arylcarbonyl, and any combination thereof, wherein the heteroaryl group is optionally substituted with one or more substituents selected from halo, trifluoromethyl, amino, alkyl, hydroxyalkyl, aryl, alkoxy, hydroxyl, carboxyl, sulfonyloxy, carboxyalkyl, sulfonyloxyalkyl, alkylcarbonyl, arylcarbonyl, and any combination thereof,

R¹⁰³ and R¹⁰ are independently selected from hydrogen, hydroxyl, amino, mercapto, ureido, C₁-C₆ alkyl carbonylamino, hydroxy C₁-C₆ alkyl, and hydrazinyl R¹⁰⁵ is selected from hydrogen, C₁-C₃ alkyl aminocarbonyl, di(Ci-C3 alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C cycloalkylamino, hydroxylamino, and C₂-C₃ alkenyl; and

R¹⁰⁶ is selected from hydrogen, C₁-C₆ alkyl, C₂-C₆ alkenyl, C₂-C₆ alkynyl, heteroaryl, and C₁-C₆ aminoalkyl;

or a pharmaceutically acceptable salt thereof,

with the proviso that, when R¹⁰³ and R¹⁰⁴ are both hydroxyl, R¹⁰⁵ is methylaminocarbonyl, R¹⁰⁶ is hydrogen, X is NHMe, and Y is CH, then Z is not iodo.

In certain embodiment for a compound of formula (I), R¹⁰⁶ is hydrogen.

In certain embodiment for a compound of formula (II), Y is N.

In certain embodiment for a compound of formula (II), R¹⁰⁵ is selected from C₁-C₃ alkyl aminocarbonyl or di(Ci-C₃ alkyl) aminocarbonyl.

In certain embodiment for a compound of formula (II), R¹⁰³ and R¹⁰⁴ are both hydroxyl.

In certain embodiment for a compound of formula (II), X is NHR¹⁰¹. In a preferred embodiment, R¹⁰¹ is C1-C6 alkyl.

In certain embodiment for a compound of formula (II), Z is

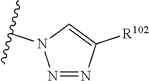

In certain embodiment for a compound of formula (II), R¹⁰² is C6-C10 aryl, wherein the aryl group is substituted with one or more substituents selected from trifluoromethyl, hydroxyalkyl, alkoxy, and any combination thereof.

In certain embodiment for a compound of formula (II), R¹⁰² is heteroaryl, and the heteroaryl group is optionally substituted with one or more substituents selected from halo, hydroxy, and alkyl.

In certain embodiment for a compound of formula (II), R¹⁰¹ is selected from C₁-C₆ alkyl, C₁-C₆ alkoxy, hydroxyl, C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C6-C14 aryl, C6-C14 aryl C1-C6 alkyl, C6-C14 diaryl C1-C6 alkyl, C6-C14 aryl C1-C6 alkoxy, heterocyclyl C₁-C₆ alkyl, heterocyclyl, and C₆-Ci₄ aryl C₃-C₈ cycloalkyl,

wherein the aryl or heterocyclyl portion of R¹⁰¹ is optionally substituted with one or more substituents each independently selected from halo, amino, hydroxyl, carboxy, alkoxycarbonyl, aminocarbonyl, alkylaminocarbonyl, dialkylaminocarbonyl, C1-C6 alkyl, C2-C6 alkenyl, C2-C6 alkynyl, C1-C6 alkoxy, C6-C14 aryloxy, hydroxy C1-C6 alkyl, hydroxy C2-C6 alkenyl, hydroxy C2-C6 alkynyl, carboxy C1-C6 alkyl, carboxy C2-C6 alkenyl, carboxy C2-C6 alkynyl, aminocarbonyl C1-C6 alkyl, aminocarbonyl C2-C₆ alkenyl, and aminocarbonyl C2-C6 alkynyl; and
wherein the alkyl or cycloalkyl portion of R¹⁰¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and arylalkoxy.

In certain embodiments for a compound of formula (II), R¹⁰⁶ is hydrogen.

In certain embodiments for a compound of formula (II), Y is N.

In certain embodiments for a compound of formula (II), R¹⁰⁵ is selected from Ci-C3 alkyl aminocarbonyl or di(Ci-C3 alkyl) aminocarbonyl.

In certain embodiments for a compound of formula (II), R¹⁰⁵ is methylaminocarbonyl.

In certain embodiments for a compound of formula (II), R¹⁰³ is hydroxyl.

In certain embodiments for a compound of formula (II), R¹⁰⁴ is hydroxyl.

In certain embodiments for a compound of formula (II), R¹⁰³ and R¹⁰⁴ are both hydroxyl.

In certain embodiments for a compound of formula (II), X is selected from NHR¹⁰¹, CH₃, and CH═C(R^{a})(R^{b}), wherein R¹⁰¹ is Ci-C₆ alkyl or C₃-C₈ cycloalkyl Ci-C₆ alkyl.

In certain embodiments for a compound of formula (II), R¹⁰¹ is methyl, ethyl, or cyclopropylmethyl.

In certain embodiments for a compound of formula (II), X is NHR¹⁰¹ and R¹⁰¹ is Ci-Ce alkyl.

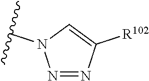

In certain embodiments for a compound of formula (II), Z is ^{N_N}

In certain embodiments for a compound of formula (II), R¹⁰² is C6-C10 aryl, wherein the aryl group is substituted with one or more substituents each independently selected from trifluoromethyl, hydroxyalkyl, and alkoxy.

In certain embodiments for a compound of formula (II), R¹⁰² is heteroaryl, and the heteroaryl group of R¹⁰² is optionally substituted with one or more substituents each independently selected from halo, hydroxy, and alkyl.

In certain embodiments for a compound of formula (II), R¹⁰² is selected from phenyl, pyridyl, pyrimidyl, pyrazinyl, pyrazolyl, furanyl, benzofuranyl, and thienyl, wherein the phenyl, pyridyl, pyrimidyl, pyrazinyl, pyrazolyl, furanyl, benzofuranyl, or thienyl of R¹⁰² is optionally substituted with one or more substituents each independently selected from halo and Ci-Ce alkyl.

In certain embodiments for a compound of formula (G), formula (IG), formula (I), or formula (H), the A3AR agonist is selected from:

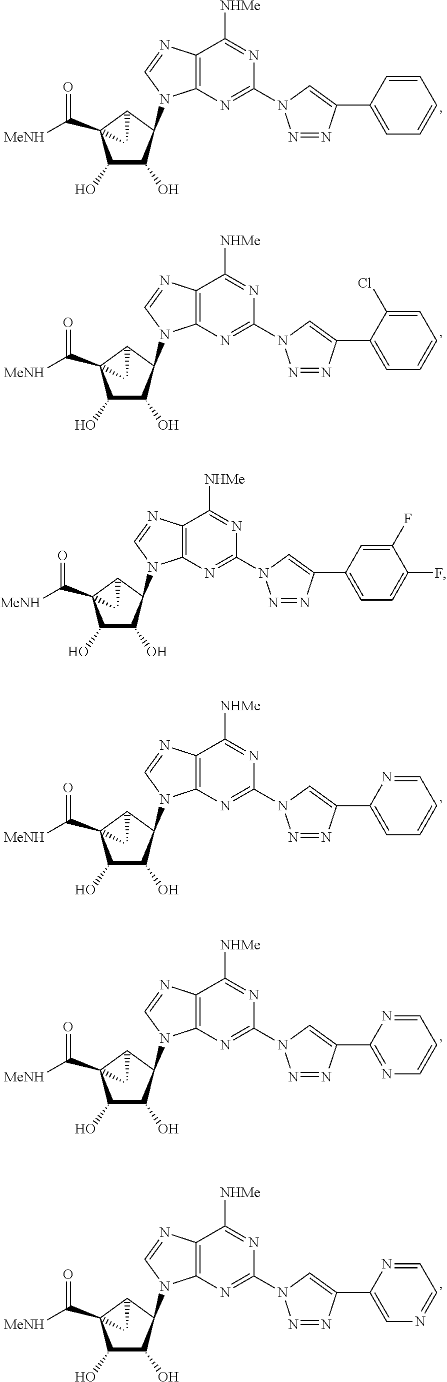

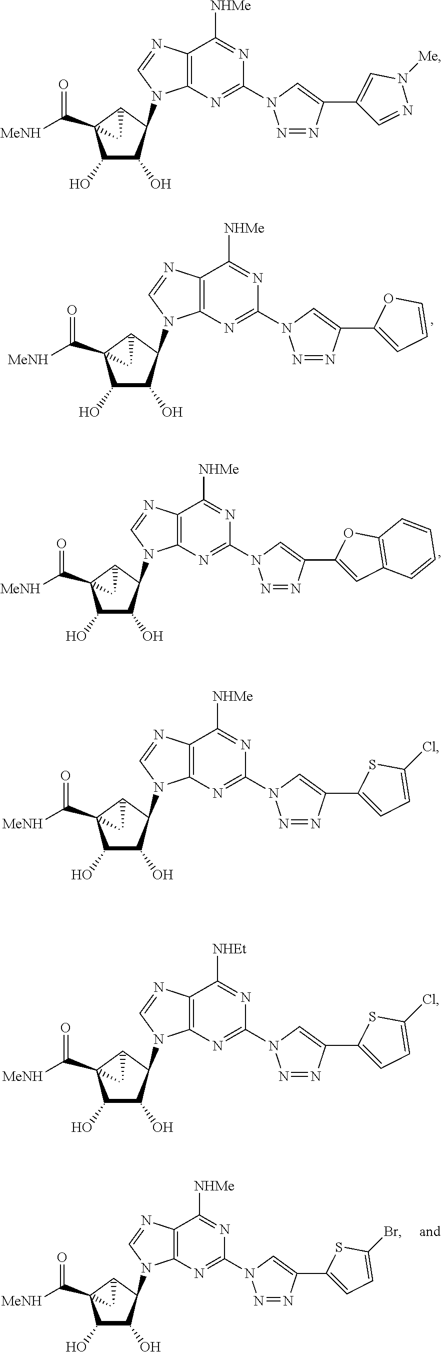

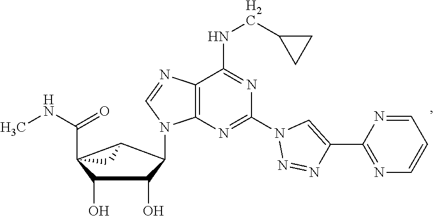

and a salt of any one thereof.

In certain preferred embodiments, the compound is selected from:

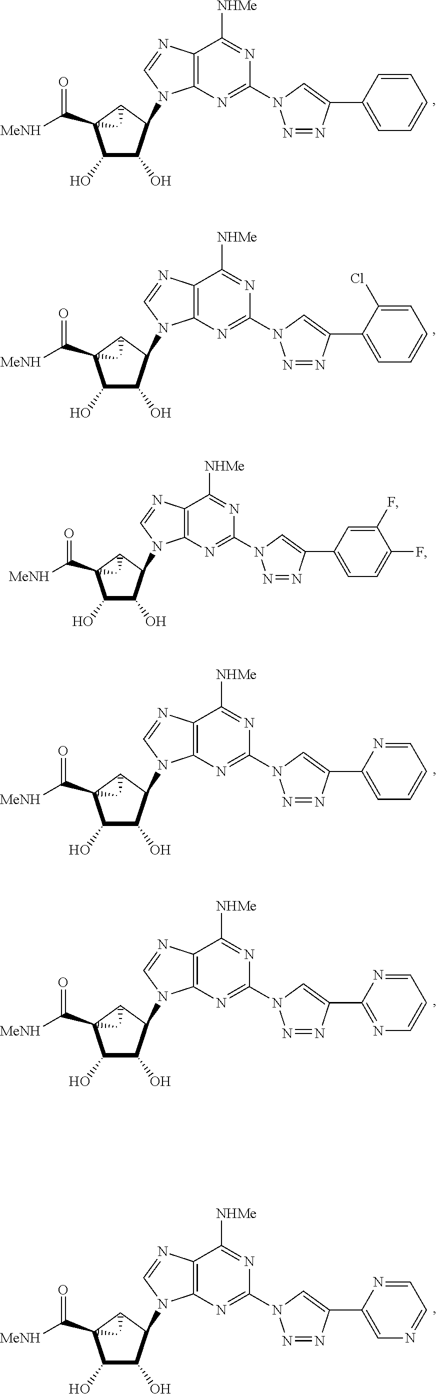

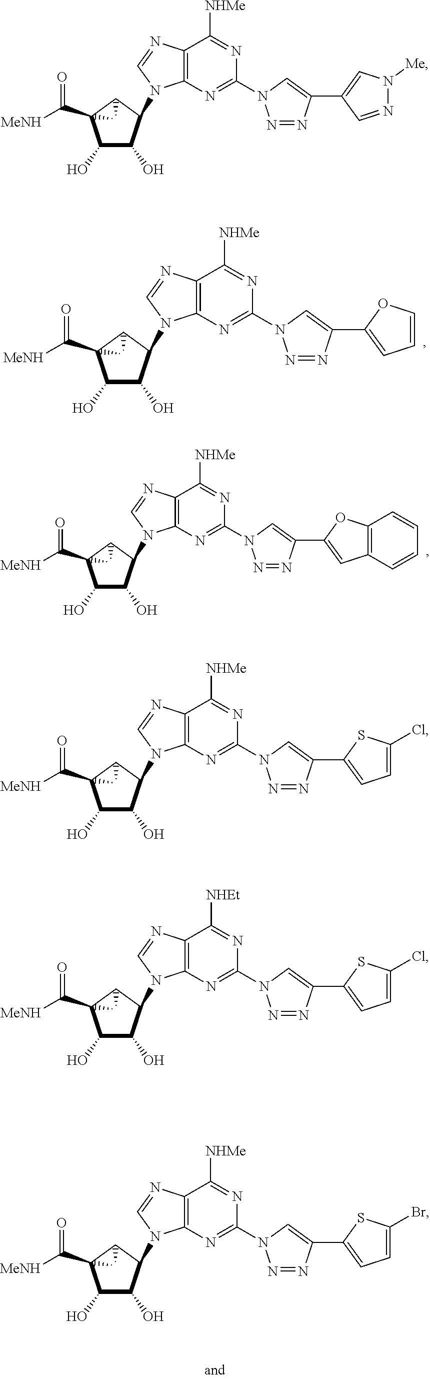

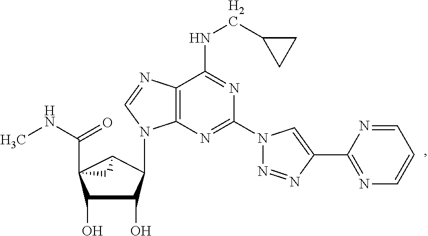

and a salt of any one thereof.

In certain embodiments, the method is for prophylactically treating said subject at risk for or suspected of having Alzheimer's disease.

According to one embodiment, the therapeutically effective amount is from 0.1 mg to 1.0 gram per day per patient (nominally weighing 60 kilograms) or equivalent amounts calculated on the basis of milligrams per kilogram of body weight, or on the basis of milligram per meter-squared of body surface area. The appropriate dosage can vary depending on the mode of administration, the particular condition to be treated and the effect desired.

Suitable subjects include non-human animals, such as, for example, nematodes, mammals, non-human primates, rodents (e.g., mice, rats, and hamsters), stock and domesticated animals (e.g., pigs, cows, sheep, horses, cats, and dogs), and birds. Particularly suitable subjects include humans. As used herein, “subject in need thereof” (also used interchangeably herein with “a patient in need thereof” and “an individual in need thereof”) refers to a subject susceptible to or at risk of a specified disease, disorder, or condition. More particularly, in the present disclosure the methods of can be used with an individual or subset of individuals who have, are susceptible to, and at elevated risk for experiencing and/or developing Alzheimer's disease. Individuals may be susceptible to or at elevated risk for these disorders or conditions due to family history, age, environment, and/or lifestyle. As used herein, “susceptible” and “at risk” refer to having little resistance to a certain disease, disorder or condition, including being genetically predisposed, having a family history of, and/or having symptoms of the disease, disorder or condition. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

The effective amount may be given via any standard drug administration method, including but not limited to injections via the intravenous, intramuscular, subcutaneous, and intrathecal routes; via inhalation (nasal or oral); per os; per rectum; or via transcutaneous methods (patches, ointments, salves, etc.).

The A3AR agonist may be formulated according to any generally known pharmaceutical method (Remington & Gennaro, 2015) that is appropriate for the intended route of administration, including any generally known and appropriate vehicle, salt or hydrate, or in any appropriate molecular precursor form (i.e., pro-drug). According to another embodiment, the A3 AR agonist is formulated in a manner intended to promote transfer across the blood-brain-barrier via any method known to one skilled in the art. The A3 AR agonist of the present disclosure can be administered to animals, preferably to mammals, and in particular to humans as therapeutics per se, as mixtures with one another or in the form of pharmaceutical preparations, and which as active constituent contains an effective dose of the compositions, in addition to customary pharmaceutically innocuous excipients and additives. The active ingredients can be introduced in a pharmaceutical composition together with one or more adjuvants, excipients, carriers, buffers, diluents, and/or other customary pharmaceutical auxiliaries. Pharmaceutically acceptable carriers, and, optionally, other therapeutic and/or prophylactic ingredients must be “acceptable” in the sense of being compatible with the other ingredients of the formulation and not harmful to the recipient thereof.

According to one embodiment, the A3AR agonist may be given on a daily basis (once, twice, three times or 4 times per day) to a patient diagnosed with Alzheimer's disease. In another embodiment, the A3AR agonist may be given on a daily basis (once, twice, three times or 4 times per day) to a patient who is suspected of being in a pre-AD state because of the presence of a biomarker for the presence of AD as a prophylactic treatment.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference:
Remington J P & Gennaro A R. Remington: The Science and Practice of Pharmacy, 2015, 22nd Edition, Mack Co., Easton Pa.
Tosh D K, et al. In vivo phenotypic screening for treating chronic neuropathic pain: modification of C2-arylethynyl group of conformationally constrained A3 adenosine receptor agonists. Journal Medicinal Chemistry (2014) 57: 9901-14.
Tosh D K, et al. Efficient, large-scale synthesis and preclinical studies of MRS5698, a highly selective A3 adenosine receptor agonist that protects against chronic neuropathic pain. Piirinergic Signalling (2015a) 11:371-387.
Tosh D K, et al. Structure-based design, synthesis by click chemistry and in vivo activity of highly selective A3 adenosine receptor agonists. Medicinal Chemistry Communications (2015b) 6: 555-63.
Tosh D K, et al. Rigidified A3 adenosine receptor agonists: 1-deazaadenine modification maintains high in vivo efficacy. ACS Medicinal Chemistry Letters (2015c) 6: 804-8.
Tosh D K, et al. Purine (N)-methanocarba nucleoside derivatives lacking an exocyclic amine as selective A3 adenosine receptor agonists. Journal Medicinal Chemistry (2016) 59: 3249-63.

1. A method for the treatment of Alzheimer's disease by administering a selective agonist for the human adenosine A3 receptor (A3 AR) subtype to a patient in need thereof, wherein: the selective agonist for the human adenosine A3 receptor (A3 AR agonist is a compound of Formula (I′): 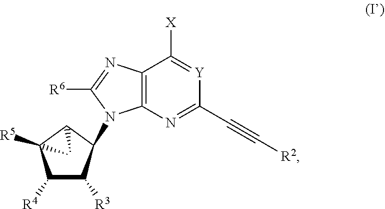
or a pharmaceutically acceptable salt thereof, wherein:
X is selected from NR^{o}R′, CH₃, and CH═C(R^{a})(R^{b}),
wherein R^{a} and R^{b} are independently selected from hydrogen, hydroxyl, C₁-C₆ alkyl, and C₆-C14 aryl;
Y is N or CH;
R^{o} is hydrogen or CfR;
R¹ is selected from hydrogen, C₁-C₆ alkyl, C₁-C₆ alkoxy, hydroxyl, C₃-C₈ cycloalkyl, C₃-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tricycloalkyl C₁-C₆ alkyl, C₆-Ci₄ aryl, C₆-Ci₄ aryl C₁-C₆ alkyl, C₆-C14 diaryl C1-C₆ alkyl, C₆-Ci4 aryl C1-C₆ alkoxy, heterocyclyl C1-C₆ alkyl, heterocyclyl, and C₆-C14 aryl C3-C8 cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C2-C₆ alkenyl, C2-C₆ alkynyl, C1-C₆ alkoxy, C₆-C14 aryloxy, hydroxy C1-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;
R² is selected from C₆-C₁₂ aryl, C₃-C₈ cycloalkyl, heteroaryl, and metallocenyl,
wherein the aryl group of R² is substituted with one or more substituents each independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl;
R³ and R⁴ are independently selected from hydrogen, hydroxyl, amino, mercapto, ureido, C₁-C₆ alkyl carbonylamino, hydroxy C₁-C₆ alkyl, and hydrazinyl;
R⁵ is selected from C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C₆ cycloalkylamino, hydroxylamino, and C₂-C₃ alkenyl; and
R⁶ is selected from hydrogen, C₁-C₆ alkyl, C₂-C₆ alkenyl, C₂-C₆ alkynyl, heteroaryl, and C₁-C₆ aminoalkyl,
with the provisos that:
when R¹ is methyl, then R² is selected from the group consisting of 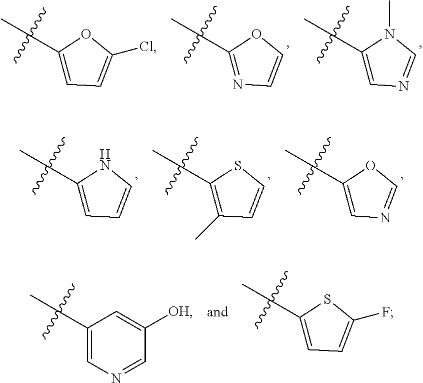
and
when R¹ is halobenzyl, diphenylethyl, or phenylethyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

2. The method of claim 1, wherein the A3AR agonist of Formula (I′) is a compound of Formula (I): 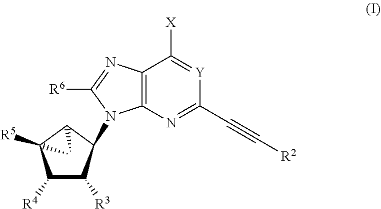
wherein:
X is selected from NHR¹, CFF, and CH═C(R^{a})(R^{b});
R¹ is selected from C1-C₆ alkyl, C1-C₆ alkoxy, hydroxyl, C3-C8 cycloalkyl, C3-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl C₁-C₆ alkyl, C₆-C₁₄ diaryl C₁-C₆ alkyl, C₆-Ci₄ aryl Ci-C₆ alkoxy, heterocyclyl C₁-C₆ alkyl, heterocyclyl, 4-[[[4-[[[(2-amino C1-C6 alkyl) amino]-carbonyl]-Ci-C6 alkyl] anilino] carbonyl]C1-C6 alkyl] C6-Ci4 aryl, and C6-Ci4 aryl C3-C8 cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, Ci-C6 alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C6 alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy; and
R² is selected from C₆-C₁₂ aryl, C₃-C₈ cycloalkyl, heteroaryl, and metallocenyl, wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkyl carbonyl, and arylcarbonyl.

3. The method of claim 1, wherein when R¹ is methyl, then R² is selected from the group consisting of

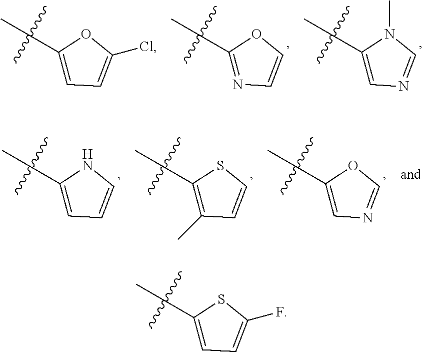

4. (canceled)

5. The method of claim 1, wherein when R¹ is halobenzyl, diphenylmethyl, diphenylethyl, or phenylethyl, then R² is thienyl, wherein the thienyl group of R² is optionally substituted with one or more halo, each independently selected.

6. The method of claim 1, wherein when R¹ is an optionally substituted C6 aryl C₁-C₂ alkyl or optionally substituted di-C6 aryl C₁-C₂ alkyl, then R² is selected from the group consisting of C₁₀-C₁₂ aryl, C₃-C₈ cycloalkyl, 5 or 6 membered heteroaryl, and metallocenyl, wherein the aryl, cycloalkyl, or heteroaryl group of R² is optionally substituted with one or more halo, each independently selected.

7. (canceled)

8. The method of claim 1, wherein:
X is selected from CTR, CH═C(R^{a})(R^{b}), and NHR¹, wherein:
R¹ is selected from C₂-C₆ alkyl, Ci-C6 alkoxy, hydroxyl, C₃-C₈ cycloalkyl, C₃-C₈ cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl Ci-C6 alkoxy, heterocyclyl C₁-C₆ alkyl, heterocyclyl, and C₆-C₁₄ aryl C₃-C₈ cycloalkyl,
wherein the aryl or heterocyclyl portion of R¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, Ci-C₆ alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C₂-C₆ alkynyl; and
wherein the alkyl or cycloalkyl portion of R¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and arylalkoxy.

9.-12. (canceled)

13. The method of claim 1, wherein when R¹ is C₂-C₃ alkyl, then R² is selected from C₆-C₁₂ aryl, C₃-C₈ cycloalkyl, heteroaryl, and metallocenyl,
wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from fluoro, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl.

14. The method of claim 1, wherein when R¹ is C₂-C₃ alkyl, then R² is selected from C₆-C₁₂ aryl, C₃-C₈ cycloalkyl, metallocenyl, methylthienyl, fluorothienyl, and 5 or 6 membered heteroaryl comprising one or more heteroatoms independently selected from N and O,
wherein the aryl group of R² is substituted with one or more substituents each
independently selected from trifluoromethyl, hydroxyalkyl, alkoxy, sulfonyloxy, carboxyalkyl, sulfonyloxyalkyl, and arylcarbonyl; and
wherein the heteroaryl group of R² is optionally substituted with one or more substituents each independently selected from halo, trifluoromethyl, amino, alkyl, hydroxyalkyl, aryl, benzo, alkoxy, hydroxyl, carboxyl, sulfonyloxy, carboxyalkyl, sulfonyloxyalkyl, alkylcarbonyl, and arylcarbonyl.

15. (canceled)

16. (canceled)

17. The method of claim 1, wherein when R¹ is methyl, R³ and R⁴ are both hydroxyl, R⁶ is hydrogen, and R⁵ is methylaminocarbonyl, R² is not 2-pyridyl or phenyl.

18. The method of claim 1, when R¹ is methyl, R³ and R⁴ are both hydroxyl, R⁶ is hydrogen, and R⁵ is methylaminocarbonyl, R² is not 2-pyridyl.

19. The method of claim 1, wherein R⁶ is hydrogen.

20. The method of claim 1, wherein Y is N.

21. The method of claim 1, wherein R⁵ is selected from C₁-C₃ alkyl aminocarbonyl or di(Ci-C₃ alkyl) aminocarbonyl.

22.-40. (canceled)

41. A method for the treatment of Alzheimer's disease by administering a selective agonist for the human adenosine A3 receptor (A3 AR) subtype to a patient in need thereof, wherein:
the selective agonist for the adenosine A3 human receptor subtype (A3AR agonist) is a compound of Formula (II′): 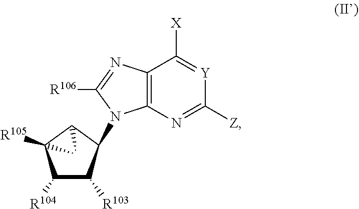
or a pharmaceutically acceptable salt thereof, wherein:
X is selected from NHR¹⁰¹, CFF, and CH═C(R^{a})(R^{b}),
wherein R^{a} and R^{b} are independently selected from hydrogen, hydroxyl, C₁-C₆ alkyl, and C₆-C14 aryl;
Y is N or CH;
R¹⁰¹ is selected from C1-C₆ alkyl, C1-C₆ alkoxy, hydroxyl, C3-C8 cycloalkyl, C3-C8 cycloalkyl C₁-C₆ alkyl, C₃-C₈ dicycloalkyl C₁-C₆ alkyl, C₇-C₁₂ bicycloalkyl, C₇-C₁₂ bicycloalkyl C₁-C₆ alkyl, C₇-C₁₄ tri cycloalkyl C₁-C₆ alkyl, C₆-Ci₄ aryl, C₆-Ci₄ aryl C₁-C₆ alkyl, C₆-C₁₄ diaryl C₁-C₆ alkyl, C₆-Ci₄ aryl C₁-C₆ alkoxy, heterocyclyl C₁-C₆ alkyl, heterocyclyl, and C₆-Ci₄ aryl C₃-C₈ cycloalkyl,
wherein the aryl or heterocyclyl portion of R¹⁰¹ is optionally substituted with one or more substituents each independently selected from halo, amino, hydroxyl, carboxy, alkoxycarbonyl, aminocarbonyl, alkylaminocarbonyl, dialkylaminocarbonyl, C₁-C₆ alkyl, C₂-C₆ alkenyl, C₂-C₆ alkynyl, C₁-C₆ alkoxy, C₆-C₁₄ aryloxy, hydroxy C₁-C₆ alkyl, hydroxy C₂-C₆ alkenyl, hydroxy C₂-C₆ alkynyl, carboxy C₁-C₆ alkyl, carboxy C₂-C₆ alkenyl, carboxy C₂-C₆ alkynyl, aminocarbonyl C₁-C₆ alkyl, aminocarbonyl C₂-C₆ alkenyl, and aminocarbonyl C₂-C₆ alkynyl; and
wherein the alkyl or cycloalkyl portion of R¹⁰¹ is optionally substituted with one or more substituents each independently selected from halo, amino, alkyl, alkoxy, aryloxy, hydroxyalkyl, hydroxyalkenyl, hydroxyalkynyl, aminocarbonylalkoxy, and aryl alkoxy;
Z is halo, azido, or a group of the formula: 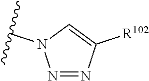
wherein:
R¹⁰² is selected from C₆-C₁₂ aryl, C₆-C₁₂ aryl-C₁-C₆ alkyl, C₃-C₈ cycloalkyl, heteroaryl, and metallocenyl,
wherein the aryl or heteroaryl group of R¹⁰² is optionally substituted with one or more substituents each independently selected from halo, trifluoromethyl, amino, alkyl, hydroxyalkyl, aryl, alkoxy, hydroxyl, carboxyl, sulfonyloxy, carboxyalkyl, sulfonyloxyalkyl, alkylcarbonyl, and arylcarbonyl;
R¹⁰³ and R¹⁰⁴ are independently selected from hydrogen, hydroxyl, amino, mercapto, ureido, C₁-C₆ alkyl carbonylamino, hydroxy C₁-C₆ alkyl, and hydrazinyl;
R¹⁰⁵ is selected from hydrogen, C₁-C₃ alkyl aminocarbonyl, di(Ci-C₃ alkyl) aminocarbonyl, C₁-C₃ alkylthio C₁-C₃ alkyl, halo C₁-C₃ alkyl, hydrazinyl, amino C₁-C₃ alkyl, hydroxy C₁-C₃ alkyl, C₃-C₆ cycloalkylamino, hydroxylamino, and C₂-C₃ alkenyl; and
R¹⁰⁶ is selected from hydrogen, C₁-C₆ alkyl, C₂-C₆ alkenyl, C₂-C₆ alkynyl, heteroaryl, and C₁-C₆ aminoalkyl,
with the proviso that, when R¹⁰³ and R¹⁰⁴ are both hydroxyl, R¹⁰⁵ is methylaminocarbonyl, R¹⁰⁶ is hydrogen, X is NHMe, and Y is CH, then Z is not iodo.

42. The method of claim 41, wherein the A3AR agonist of Formula (II′) is a compound of Formula (II): 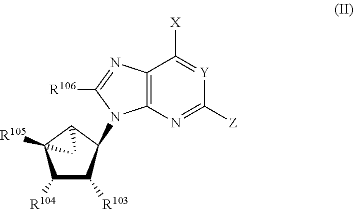
wherein:
R¹⁰² is selected from C₆-C₁₂ aryl, C₆-C₁₂ aryl-C₁-C₆ alkyl, C₃-C₈ cycloalkyl, heteroaryl, and metallocenyl,
wherein the aryl group of R¹⁰² is optionally substituted with one or more substituents each independently selected from trifluoromethyl, hydroxyalkyl, alkoxy, sulfonyloxy, carboxyalkyl, sulfonyloxyalkyl, and arylcarbonyl; and
wherein the heteroaryl group of R¹⁰² is optionally substituted with one or more substituents each independently selected from halo, trifluoromethyl, amino, alkyl, hydroxyalkyl, aryl, alkoxy, hydroxyl, carboxyl, sulfonyloxy, carboxyalkyl, sulfonyloxyalkyl, alkylcarbonyl, and arylcarbonyl.

43.-46. (canceled)

47. The method of claim 41, wherein R¹⁰³ is hydroxyl.

48. The method of claim 41, wherein R¹⁰⁴ is hydroxyl.

49. (canceled)

50. The method of claim 41, wherein X is selected from NHR¹⁰¹, CH₃, and CH═C(R^{a})(R^{b}), wherein R¹⁰¹ is C₁-C₆ alkyl or C₃-C₈ cycloalkyl C₁-C₆ alkyl.

51. (canceled)

52. (canceled)

53. The method of claim 41, wherein Z is

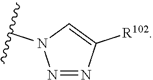

54. The method of claim 53, wherein R¹⁰² is C₆-C₁₀ aryl, wherein the aryl group is substituted with one or more substituents each independently selected from trifluoromethyl, hydroxyalkyl, and alkoxy.

55. The method of claim 53, wherein R¹⁰² is heteroaryl, and the heteroaryl group of R¹⁰² is optionally substituted with one or more substituents each independently selected from halo, hydroxy, and alkyl.

56.-59. (canceled)
